(19) United States
(12) Patent Application Publication
(10) Pub. No.: US 2009/0209485 A1
(43) Pub. Date: Aug. 20, 2009

(54) MODULATING ANGIOGENESIS WITH NOD FACTORS SUCH AS GLUCOSAMINE OLIGOSACCHARIDES

(76) Inventors: Christopher Richard Parish, Campbell (AU); Michael A. Djordjevic, Kingston (AU); Barry G. Rolfe, Curtin (AU); Peter M. Gresshoff, St. Lucia (AU)

Correspondence Address: SEED INTELLECTUAL PROPERTY LAW GROUP PLLC, 701 FIFTH AVE, SUITE 5400, SEATTLE, WA, 98104, US

(21) Appl. No.: 11/910,166

(22) PCT Filed: May 31, 2006
(86) PCT No.: PCT/AU06/00432

§ 371 Date: Sep. 9, 2008

Related U.S. Application Data
(60) Provisional application No. 60/667,091, filed on Apr. 1, 2005.

(51) Int. Cl. A61K 31/715; A61P 35/00; A61P 35/04

(52) U.S. Cl. 514/54

(57) ABSTRACT

This invention relates to the use of Nod factors and derivatives thereof for the modulation of blood vessel growth and development as well as compositions for modulating angiogenesis.

### FIELD OF THE INVENTION

This invention relates to the use of Nod factors and derivatives thereof for the modulation of blood vessel growth and development.

### BACKGROUND OF THE INVENTION

Angiogenesis refers to the process in which new blood vessels arise from pre-existing vessels. The process occurs under both normal physiological conditions and in pathological situations. Physiological angiogenesis is associated with normal blood vessel development in the foetus whereas pathological angiogenesis occurs in important disease states such cancer, ischemic heart disease, diabetes, chronic inflammation and aberrant wound healing (Folkman J., Semin. Oncol., 2002, 29, 15; Carmeliet, P., Nat. Med., 2003, 9, 653-60; Dvorak, H. F., Am. J. Pathol., 2003, 162, 1747-57). Many of these syndromes have been generally referred to as angiogenesis-dependent diseases. In addition, angiogenesis is known to be tightly regulated by numerous endogenous anti-angiogenic and pro-angiogenic factors. Thus, approaches that target angiogenesis in a range of disease have enormous therapeutic potential (Kerbel R, Folkman J., Nat Rev Cancer. 2002, 2, 727-39; Soria J. C., Fayette J, Armand J P., Ann Oncol. 2004, 15 Suppl 4, 223-7).

A considerable number of angiogenesis inhibitors have been identified and many have already entered clinical trials (Soria, J. C., Fayette J., Armand, J. P., Ann. Oncol., 2004, 15 Suppl 4, 223-7). The first anti-angiogenic drug to be registered by the FDA was Bevacizumab, a humanised monoclonal antibody (mAb) against vascular endothelial growth factor (VEGF), a key growth factor involved in initiating angiogenesis. Additional anti-angiogenic drugs at advanced stages of development include tyrosine kinase inhibitors that block VEGF receptor signalling by VEGF, mAbs that block the interaction of VEGF with VEGF receptors, cyclo-oxygenase inhibitors, endogenous polypeptide inhibitors (eg. angiostatin, endostatin), epidermal growth factor receptor antagonists, integrin antagonists, heparan sulfate mimetics (eg. PI-88), estrogen metabolites and even old drugs developed for other purposes (eg. thalidomide). Although inhibition of solid tumour growth is the major clinical target of these anti-angiogenic drugs, they can be used in other disease situations such as inhibition of diabetic retinopathy and chronic inflammation. Recently angiogenesis inhibitors have also been used to induce adipose loss in obese animals: see, Rupnick, M. A., Panigrahy, D., Zhang, C. Y., Dallabrida, S. M., Lowell, B. B., Langer, R., Folkman, M., J., Proc Natl Acad Sci USA., 2002, 99, 10730-5. Other classes of molecules, such as chitosans, have recently shown marginal activity as angiogenesis inhibitors, see: Prashanth, K. V. H., and Tharanathan, R. N., Biochimica and Biophysica Acta, 2005, 1722, 22-29.

Inducing angiogenesis is desirable in situations where vascularisation is to be established or extended, for example, after tissue or organ transplantation or to stimulate establishment of collateral circulation in tissue infarction or arterial stenosis, such as in coronary heart disease and thromboangitis obliterans. Angiogenic growth factors/growth factor receptor agonists could be used to assist wound healing and in treating ischemic conditions, including cardiovascular and limb ischemia.

Nodulation (Nod) factors are key signalling molecules that play a pivotal role during initiation of nodule development and bacterial development. They are produced by rhizobia, which nodulate specific leguminous host plants and the nonlegume Parasponia. Such symbioses between rhizobia and plant result in the formation of root nodules, new organs occupied by differentiated bacteria, that fix atmospheric nitrogen and provide it to their respective host plant, thereby promoting plant growth independently of the available soil nitrogen. Nod factors consist of an oligomeric backbone of β(1→4)-linked N-acetyl-D-glucosaminyl residues, N-acylated with aliphatic chains at the non-reducing terminal residue affording lipochitooligosaccharides. Generally, Nod factors differ as follows: the number of GlcNAc residues present in the chitooligosaccharide backbone, the nature of the fatty acyl substituent, and the substituents at the non-reducing and/or reducing terminal residues. However, Nod factors may also be substituted at non-terminal residues see D'Haeze, W., and Holsters, M., Glycobiology, 2002, 12(6), 79R-105R.

Various Nod factors have been previously described in the prior art, see Price, N. P., et al., Mol. Microbiol., 1992, 23, 3575-84; U.S. Pat. No. 5,646,018; U.S. Pat. No. 5,549,718; Roche, P., J. Biol. Chem., 1991, 266, 10933-10940; Nathalie, Fabienne, D-C., Plant Physiol., 1999; 120(1), 83-92; and Carlson R W et al., J. Biol. Chem., 1993, 268, 18372-18381. It has now been surprisingly found that Nod factors are useful agents for modulating angiogenic states. Currently, most angiogenesis therapies are directed towards finding antibodies or drugs that affect angiogenesis. As antibodies are proteins, these therapies run the risk of generating immune responses in recipients whereas small oligosaccharides are regarded as being less likely to be recognised adversely by the immune system. In addition, small oligosaccharides may be less likely to induce toxic effects than other classes of drugs (such as hormone derivatives). For example, one drug being trialed as an anti-angiogenic factor at the moment is Thalidomide, which is known to cause birth defects. Accordingly, methods for inducing or inhibiting angiogenesis with Nod factors and derivatives thereof are disclosed.

### SUMMARY OF THE INVENTION

In a first aspect, the invention provides a method of modulating angiogenesis in a mammal comprising administering to the mammal a therapeutically effective amount of a Nod factor or derivative thereof.

In one embodiment the invention provides a method of modulating angiogenesis in a mammal comprising administering to the mammal a therapeutically effective amount of a oligosaccharide of formula I or a pharmaceutically acceptable salt thereof:

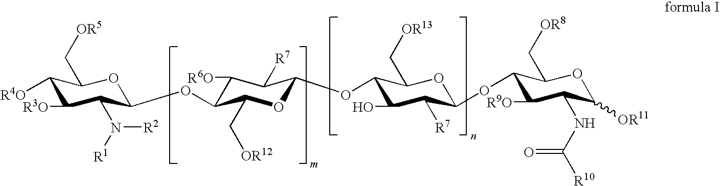

wherein:
R¹ is hydrogen, -X-Alk or -X-Alk¹-Q-Y-Alk²;
wherein:
X is selected from —C(O)—, —C(NR^{N})—, —C(S)—, —SO₂—, —P(O)(OR^{N})— wherein R^{N} is hydrogen, hydroxy, amino, optionally substituted C₁₋₈alkyl, optionally substituted C₂₋₈alkenyl, optionally substituted C₂₋₈alkynyl, optionally substituted C₁₋₄alkylaryl, and optionally substituted aryl;
Alk is selected from an optionally substituted, straight chain or branched, alkyl, alkenyl or alkynyl group having from 2 to 30 carbon atoms;
Alk¹ is absent or present and is selected from an optionally substituted divalent C₁₋₁₀alkyl, optionally substituted divalent C₂₋₁₀alkenyl and optionally substituted divalent C₂₋₁₀alkynyl chain;
Q is absent or present and is selected from an optionally substituted divalent cycloalkyl, optionally substituted divalent cycloalkenyl, optionally substituted divalent heterocycle, optionally substituted divalent aryl or optionally substituted divalent heteroaryl ring system;
Y is absent or present and is selected from —NH—, —O—, —S—, —NHC(O)—, —C(O)NH—, NHSO₃—, —C(R^{G})═N—N—, —NHC(O)NH—, —NHC(S)NH—, —NHC(NH)NH—, —C(R^{G})═N— and —N═C(R^{G})—, wherein R^{G} is hydrogen, optionally substituted C₁₋₆alkyl, optionally substituted arylC₁₋₄alkyl, optionally substituted aryl or optionally substituted heteroaryl, provided that both Q and Y are not simultaneously absent; and
Alk² is absent or present and is selected an optionally substituted, straight chain or branched, alkyl, alkenyl or alkynyl group having from 1 to 30 carbon atoms.
   R² is hydrogen, C₁₋₄alkyl or R² can combine with R¹ and N to form an azide;
   R³ and R⁴ are independently selected from hydrogen, carbamoyl and C₁₋₄acyl;
   R⁵ is hydrogen, carbamoyl, fucopyranosyl and C₁₋₄acyl;
   R⁶ is hydrogen, C₁₋₄acyl or a monosaccharide;
   R⁷ is independently selected from an acetamide or a hydroxyl group;
   R⁸ is hydrogen, sulphonato, C₁₋₄acyl or a monosaccharide;
   R⁹ is hydrogen or a monosaccharide;
   R¹⁰ is hydrogen or optionally substituted C₁₋₄alkyl;
   R¹¹ is hydrogen, a monosaccharide, glycerol, C₁₋₄acyl or C₁₋₄ alkyl;
   R¹² is hydrogen, fucopyranosyl or C₁₋₄acyl;
   R¹³ is independently selected from hydrogen or fucopyranosyl;
   m is an integer selected from 0 and 1;
   n is an integer selected from 0 to 3; and
   where the reducing end sugar ring is in open chain or ring closed form.

As used herein, the term “optionally substituted” means that a group may include one or more substituents that do not interfere with the biological activity of the compound of formula I. In some instances, the substituent may be selected to improve certain physico-chemical properties such as solubility under physiological conditions. Examples of optional substituents include halo, C₁₋₄alkyl, C₂₋₄alkenyl, C₂₋₄alkynyl, C₁₋₄alkoxy, haloC₁₋₄alkyl, hydroxyC₁₋₄alkyl, C₁₋₄alkoxy, C₁₋₇acyl, C₁₋₇acyloxy, hydroxy, aryl, amino, azido, nitro, nitroso, cyano, carbamoyl, trifluoromethyl, mercapto, C₁₋₄alkylamino, C₁₋₄dialkylamino, aryloxy, formyl, carbamoyl, C₁₋₆alkylsulphonyl, C₁₋₆arylsulphonyl, C₁₋₆alkylsulphonamido, C₁₋₆arylsulphonamido, C₁₋₄alkylamino, di(C₁₋₄alkyl)amino, and C₁₋₄alkoxycarbonyl.

A “divalent” chemical moiety, refers to a chemical moiety which needs two hydrogen atoms in order to be an independent and preferably stable molecule. Thus, a diradical has two open valence sites on one or two atoms, through which the diradical may be bonded to other atom(s).

The term “heterocycle” as used herein, refers to mono or bicyclic rings or ring systems which include at least one heteroatom atom selected from nitrogen, sulphur and oxygen. The rings or ring systems generally include 1 to 9 carbon atoms in addition to the heteroatom(s) and may be saturated, unsaturated, aromatic or pseudoaromatic. Aromatic and psuedoaromatic heterocycles may be termed heteroaromatic or heteroaryl rings. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro [2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinoyl and the like, each of which may be optionally substituted with C₁₋₆acyl, C₁₋₆alkyl, C₁₋₆alkoxy, C₂₋₆alkenyl, C₂₋₆alkynyl, C₁₋₆alkylsulphonyl, arylsulphonyl, C₁₋₆alkylsulphonamido, halo, hydroxy, mercapto, trifluoromethyl, amino, azido, nitro, cyano, carbamoyl, aminocyano, or mono or di(C₁₋₆alkyl)amino. Also included are fused ring and spiro compounds containing, for example, the above heterocycles.

The term “cycloalkyl” as used herein, refers to a non-aromatic mono- or multicyclic ring system of about 3 to about 10 carbon atoms, preferably of about 5 to about 10 carbon atoms. Preferred ring sizes of monocyclic ring systems include about 5 to about 6 ring atoms. The cycloalkyl is optionally substituted with one or more substituents which may be the same or different, and are as defined herein. Examples of monocyclic cycloalkyl groups include cyclopropyl, cyclobutyl, cyclopentyl, cyclohexyl, cycloheptyl, and the like. Exemplary multicyclic cycloalkyl include [3.3.0]bicyclooctane, [4.3.0]bicyclononane, [4.4.0]bicyclodecane (decalin), [2.2.2]bicyclooctane, norbornyl, adamant-(1- or 2-)yl, and the like.

As used herein “cycloalkenyl” refers to a non-aromatic mono- or multicyclic ring system of about 3 to about 10 carbon atoms, preferably of about 5 to about 10 carbon atoms, and which contains at least one carbon-carbon double bond. Preferred ring sizes monocyclic ring systems include about 5 to about 6 ring atoms. The cycloalkenyl is optionally substituted with one or more substituents which may be the same or different, and are as defined herein. Exemplary monocyclic cycloalkenyl include cyclopentenyl, cyclohexenyl, cycloheptenyl, and the like.

As used herein, the term “aryl” refers to optionally substituted monocyclic, bicyclic, and biaryl carbocyclic aromatic groups, of 6 to 14 carbon atoms, covalently attached at any ring position capable of forming a stable covalent bond, certain preferred points of attachment being apparent to those skilled in the art. Examples of monocyclic aromatic groups include phenyl, toluoyl, xylyl and the like, each of which may be optionally substituted with C₁₋₆acyl, C₁₋₆alkyl, C₁₋₆alkoxy, C₂₋₆alkenyl, C₁₋₆alkynyl, C₁₋₆alkylsulphonyl, arylsulphonyl, C₁₋₆alkylsulphonamido, arylsulphonamido, halo, hydroxy, mercapto, trifluoromethyl, carbamoyl, amino, azido, nitro, cyano, C₁₋₆alkylamino or di(C₁₋₆alkyl)amino. Examples of bicyclic aromatic groups include 1-naphthyl, 2-naphthyl, indenyl and the like, each of which may be optionally substituted with C₁₋₆acyl, C₁₋₆alkyl, C₁₋₆alkoxy, C₂₋₆alkenyl, C₂₋₆alkynyl, C₁₋₆alkylsulphonyl, arylsulphonyl, C₁₋₆alkylsulphonamido, arylsulphonamido, halo, hydroxy, mercapto, trifluoromethyl, carbamoyl, amino, azido, nitro, cyano, C₁₋₄alkylamino or di(C₁₋₆alkyl)amino. Examples of biaryl aromatic groups include biphenyl, fluorenyl and the like, each of which may be optionally substituted with C₁₋₆acyl, C₁₋₆alkyl, C₁₋₆alkoxy, C₂₋₆alkenyl, C₂₋₆alkynyl, C₁₋₆alkylsulphonyl, arylsulphonyl, C₁₋₆alkylsulphonamido, arylsulphonamido, halo, hydroxy, mercapto, trifluoromethyl, carbamoyl, amino, azido, nitro, cyano, C₁₋₆alkylamino or di(C₁₋₆alkyl)amino.

As used herein, the term “C₁₋₆alkyl”, as used alone or as part of a group such as “di(C₁₋₆alkyl)amino”, refers to straight chain, branched or cyclic alkyl groups having from 1 to 6 carbon atoms. Examples of such alkyl groups include methyl, ethyl, n-propyl, isopropyl, n-butyl, cyclopentyl and cyclohexyl. Similarly, C₁₋₄, C₁₋₈, C₁₋₁₀ and C₁₋₃₀ alkyl, for example, refer to groups having 1 to 4, 1 to 8, 1 to 10 and 1 to 30 carbon atoms, respectively.

As used herein, the terms “C₁₋₆alkoxy” and “C₁₋₆alkyloxy” refer to straight chain or branched alkoxy groups having from 1 to 6 carbon atoms. Examples of C₁₋₆alkoxy include methoxy, ethoxy, n-propoxy, isoptopoxy, cyclohexyloxy, and the different butoxy isomers. Similarly, C₁₋₄, C₁₋₈ and C₁₋₁₀ alkoxy refer to groups having 1 to 4, 1 to 8, and 1 to 10 carbon atoms, respectively.

As used herein, the terms “C₁₋₁₀acyl” refers to straight chain or branched, aromatic or aliphatic, saturated or unsaturated acyl groups having from 1 to 10 carbon atoms. Examples of C₁₋₁₀acyl include formyl, acetyl, propionyl, butanoyl, pentanoyl, pivaloyl, benzoyl and 2-phenylacetyl, Similarly, C₁₋₄, C₁₋₆ and C₁₋₈ acyl refer to groups having 1 to 4, 1 to 6, and 1 to 8 carbon atoms, respectively.

As used herein, the term “C₂₋₈alkenyl” refers to groups formed from C₂₋₈ straight chain, branched or cyclic alkenes. Examples of C₂₋₈alkenyl include allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1,3-butadienyl, 1-4, pentadienyl, 1,3-cyclopentadienyl, 1,3-hexadienyl, 1,4-hexadienyl, 1,3-cyclohexadienyl and 1,4-cyclohexadienyl. Similarly, C₂₋₄, C₂₋₆ C₂₋₁₀ and C₂₋₂₉ alkenyl, for example, refer to groups having 2 to 4, 2 to 6, 2 to 10 and 2 to 29 carbon atoms, respectively.

As used herein, the term “C₂₋₈alkynyl” refers to groups formed from C₂₋₈ straight chain or branched groups as previously defined which contain a triple bond. Examples of C₂₋₈alkynyl include 2,3-propynyl and 2,3- or 3,4-butynyl. Similarly, C₂₋₄, C₂₋₆, C₂₋₁₀ and C₂₋₂₉ alkynyl, for example, refer to groups having 2 to 4, 2 to 6, 2 to 10 and 2 to 29 carbon atoms, respectively.

As used herein, the term “arylC₁₋₄alkyl” refers to groups formed from C₁₋₄ straight chain, branched alkanes substituted with an aromatic ring. Examples of arylC₁₋₄alkyl include methylphenyl (benzyl), ethylphenyl, propylphenyl and isopropylphenyl.

As used herein, the term “C₁₋₆alkylsulphonyl” refers to a “C₁₋₆alkyl” group attached through a sulphonyl bridge. Examples of “C₁₋₆alkylsulfonyl” groups include methylsulphonyl, ethylsulphonyl, isopropylsulphonyl and the like.

As used herein, the term “arylsulphonyl” refers to an “aryl” group attached through a sulphonyl bridge. Examples of “arylsulfonyl” groups include phenylsulphonyl, 4-methylphenylsulphonyl, 3-fluorophenylsulphonyl, 4-nitrophenylsulphonyl, naphthylsulphonyl, biphenylsulphonyl and the like.

As used herein, the term “C₁₋₆alkylsulphonamido” refers to a “C₁₋₆alkylsulphonyl” group wherein the “C₁₋₆alkylsulphonyl” group is in turn attached through the nitrogen atom of an amino group. Examples of “C₁₋₆alkylsulphonamido” groups include methylsulphonamido, ethylsulphonamido and the like.

As used herein, the term “arylsulphonamido” refers to an “arylsulphonyl” group wherein the “arylsulphonyl” is in turn attached through the nitrogen atom of an amino group. Examples of “arylsulphonamido” groups include phenylsulphonamido, 4-methylphenylsulphonamido, 3-fluorophenylsulphonamido, 4-nitrophenylsulphonamido, naphthylsulphonamido, biphenylsulphonamido and the like.

As used herein, the term “C₁₋₆alkylamino” refers to a “C₁₋₆alkyl” group attached through an amine bridge. Examples of “C₁₋₆alkylamino” include methylamino, ethylamino, butylamino and the like.

As used herein, the term “di(C₁₋₆alkyl)amino” refers to two “C₁₋₆alkyl” groups having the indicated number of carbon atoms attached through an amine bridge. Examples of “di(C₁₋₆alkyl)amino” include diethylamino, N-propyl-N-hexylamino, N-cyclopentyl-N-propylamino and the like.

As used herein term “C_{18:1}” and variations such as “C18:1” refers to an 18 carbon acyl group with a single double bond located in the chain. Similarly, the term “C_{16:2}” and like terms such as “C16:2” refers to a 16 carbon acyl group with 2 double bonds located in the chain.

As defined herein the term “saturated or unsaturated, branched or linear C₁₋₃₀acyl” refers to a substituent of formula R^{AC}—C(O)— wherein R^{AC} is a optionally substituted, straight chain or branched, alkyl, alkenyl or alkynyl group having from 1 to 30 carbon atoms. Such acyl substituents may be optionally substituted, for example with one or more hydroxy, alkyl, alkoxy or halo groups. C₁₋₃₀acyl substituents may be derived from corresponding fatty acids, such as: saturated fatty acids, monoenoic and polyenoic fatty acids, polyunsaturated fatty acids, polyunsaturated fatty acids, alpha-hydroxy fatty acids, di-hydroxy fatty acids, alpha-methoxy fatty acids, halogenated fatty acids, mono- or multi-branched fatty acids, branched hydroxy fatty acids, branched methoxy fatty acids, and ring containing fatty acids. Examples of fatty acids include: tetradecanoic acid, tetradecenoic acids, tetradecadienoic acids, hydroxy-tetradecenoic acids, methyl-tetradecenoic acids, hexadecenoic acids, hexadecenoic acids, hexadecadienoic acids, hexadecatrienoic acids, methyl-hexadecanoic acids, methyl-hexadecenoic acids, octadecanoic acids, hydroxy-octadecanoic acids, di-hydroxy-octadecanoic acids, octadecenoic acids, octadecadienoic acids, octadecatrienoic acids, octadecatetraenoic acids, eicosanoic acids, eicosaenoic acids, eicosadienoic acids, eicosatrienoic acids, eicosatetraenoic acids, hydroxy-eicosaenoic acids, docosanoic acids, docosenoic acids, docosadienoic acids, hydroxy-docosenoic acids, tetracosanoic acids, tetracosenoic acids, hexacosanoic acids, hexacosenoic acids, cyclopent-1-ene-1-tetradecanoic acids, cyclopent-2-ene-1-tetradecanoic acids, cyclopent-3-ene-1-tetradecanoic acids, cyclopentane-1-tetradecenoic acids, including: butyric acid (butanoic acid), caproic acid (hexanoic acid), caprylic acid (octanoic acid), capric acid (decanoic acid), lauric acid (dodecanoic acid), palmitoleic acid (9-hexadecenoic acid), oleic acid (9-octadecenoic acid), vaccenic acid (11-octadecenoic acid), linoleic acid (9,12-octadecadienoic acid), alpha-linolenic Acid (ALA) (9,12,15-octadecatrienoic acid), gamma-linolenic acid (GLA) (6,9,12-octadecatrienoic acid), arachidic acid (eicosanoic acid), gadoleic acid (9-cicosenoic acid), arachidonic acid (AA) 5,8,11,14-eicosatetraenoic acid, EPA (5,8,11,14,17-eicosapentaenoic acid), behenic acid (docosanoic acid), erucic acid (13-docosenoic acid), DHA (4,7,10,13,16,19-docosahexaenoic acid), and lignoceric acid (tetracosanoic acid).

Examples of straight chain or branched, optionally substituted, alkyl, alkenyl or alkynyl group having from 1 to 30 carbon atoms include: methyl, ethyl, propyl, isopropyl, butyl, sec-butyl, butenyl, pentyl, pentenyl, hexyl, hexenyl, heptyl, heptenyl, octyl, octeneyl, nonyl, nonenyl, decyl, decenyl, undecanyl, undecenyl, dodecanyl, dodeceneyl, tetradecanyl, tetradecenyl, tetradecadienyl, hydroxy-tetradecenyl, methyl-tetradeceny, hexadecenyl, hexadecadienyl, hexadecatrienyl, methyl-hexadecanyl, methyl-hexadecenyl, octadecanyl, hydroxy-octadecanyl, di-hydroxy-octadecanyl, octadecenyl, octadecadienyl, octadecatrienyl, octadecatetraenyl, eicosanyl, eicosaenyl, eicosadienyl, eicosatrieyl, eicosatetraenyl, hydroxy-eicosaenyl, docosanyl, docosenyl, docosadienyl, hydroxy-docosenyl, tetracosanyl, tetracosenyl, hexacosanyl, and hexacosenyl and the like.

As used herein the term monosaccharide refers to polyhydroxy aldehydes H—[CHOH]ᵤ—CHO or polyhydroxy ketones H—[CHOH]ᵤ—CO—[CHOH]ᵥ—H with three or more carbon atoms. The generic term ‘monosaccharide’ includes aldoses, dialdoses, aldoketoses, ketoses and diketoses, as well as deoxy sugars and amino sugars, and their derivatives, provided that the parent compound has a carbonyl group or potential carbonyl group. Monosaccharides with an aldehydic carbonyl or potential aldehydic carbonyl group are called aldoses; those with a ketonic carbonyl or potential ketonic carbonyl group, ketoses. The term ‘potential aldehydic carbonyl group’ refers to the hemiacetal group arising from ring closure. Likewise, the term ‘potential ketonic carbonyl group’ refers to the hemiketal structure. Cyclic hemiacetals or hemiketals of sugars with a five-membered (tetrahydrofuran) ring are called furanoses, those with a six-membered (tetrahydropyran) ring pyranoses. Monosaccharides containing two (potential) aldehydic carbonyl groups are called dialdoses. Monosaccharides containing two (potential) ketonic carbonyl groups are termed diketoses. Monosaccharides containing a (potential) aldehydic group and a (potential) ketonic group are called ketoaldoses. Monosaccharides in which an alcoholic hydroxy group has been replaced by a hydrogen atom are called deoxy sugars. Monosaccharides in which an alcoholic hydroxy group has been replaced by an amino group are called amino sugars. When the hemiacetal hydroxy group is replaced, the compounds are called glycosylamines. The polyhydric alcohols arising formally from the replacement of a carbonyl group in a monosaccharide with a CHOH group are termed alditols. Monocarboxylic acids formally derived from aldoses by replacement of the aldehydic group by a carboxy group are termed aldonic acids. Oxo carboxylic acids formally derived from aldonic acids by replacement of a secondary CHOH group by a carbonyl group are called ketoaldonic acids. Monocarboxylic acids formally derived from aldoses by replacement of the CH₂OH group with a carboxy group are termed uronic acids. The dicarboxylic acids formed from aldoses by replacement of both terminal groups (CHO and CH₂OH) by carboxy groups are called aldaric acids. The monosaccharides may be in D or L form. Particular examples of monosaccharides are provided as follows: an example of an aldotriose is glyceraldehyde; examples of aldotetraoses are erythrose and threose; examples of pentoses are ribose, arabinose, xylose and lyxose, examples of hexoses are allose, altrose, glucose, mannose, gulose, idose, galactose and talose, examples of aminosugars are N-acetyl-glucosamine, N-acetyl-galactosamine, and N-acetyl-mannosamine; an example of a deoxy sugar is fucose, an example of a ketopentose is ribulose, and example of a ketohexose is fructose, examples of uronic acids are galacturonic acid, glucuronic acid and iduronic acid, other carboxylic acid containing monosaccharides are sialic acid and KDO.

In preferred embodiments of the invention, one or more of the following definitions may apply:

preferably R¹ is hydrogen, —X-Alk or —X-Alk¹-Q-Y-Alk²;
preferably Alk is an optionally substituted, straight chain or branched, alkyl, alkenyl or alkynyl group having from 6 to 25 carbon atoms; more preferably Alk is an optionally substituted, straight chain or branched, alkyl, alkenyl or alkynyl group having from 10 to 25 carbon atoms; even more preferably Alk is an optionally substituted, straight chain or branched, alkyl, alkenyl or alkynyl group having from 14 to 22 carbon atoms;
preferably X is —C(O)—, —SO₂—, —P(O)(ORN)—; more preferably X is —C(O)—;
preferably Alk¹ is divalent C₁₋₄alkyl or is absent;
preferably Q is optionally substituted divalent aryl or heteroaryl, more preferably Q is optionally substituted divalent aryl; even more preferably Q is optionally substituted divalent phenyl;
preferably Y is —O—, —NH—, —S—, —NHC(O)—, or —C(O)NH—, more preferably Y is —O—, —NH—, —NHC(O)—, or —C(O)NH—;
preferably Alk² is an optionally substituted, straight chain or branched, alkyl, alkenyl or alkynyl group having from 1 to 25 carbon atoms; more preferably Alk² is an optionally substituted, straight chain or branched, alkyl, alkenyl or alkynyl group having from 5 to 25 carbon atoms; even more preferably Alk² is an optionally substituted, straight chain or branched, alkyl, alkenyl or alkynyl group having from 10 to 20 carbon atoms preferably R² is hydrogen or methyl;
preferably R³, R⁴ and R⁵ are independently selected from hydrogen, carbamoyl or acetyl, and more preferably R³ and R⁴ are independently selected from carbamoyl and hydrogen, and R⁵ is hydrogen;
preferably R⁶ is hydrogen, acetyl or fucopyranosyl, and more preferably R⁶ is hydrogen;
preferably R⁷ is an acetamide;
preferably R⁹ is hydrogen, sulphonato, C₁₋₄acyl, an unsubstituted monosaccharide, or a substituted monosaccharide of formula III:

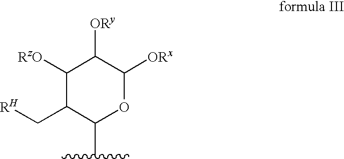

wherein:
R^{x} is hydrogen, C₁₋₄alkyl or C₁₋₄acyl, R^{y} is hydrogen, sulphonato or C₁₋₄acyl, R^{z} is hydrogen, C₁₋₄alkyl or C₁₋₄acyl, and R^{H} is H or OR^{P}, wherein R^{P} is hydrogen, C₁₋₄alkyl or C₁₋₄acyl; and
more preferably R⁸ is hydrogen, arabinosyl, sulphonato, C₁₋₄acyl or a substituted monosaccharide of formula IV:

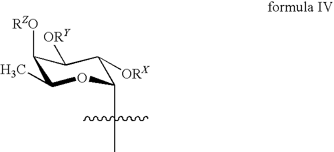

wherein:
R^{X} is hydrogen, C₁₋₄alkyl or C₁₋₄acyl, R^{Y} is hydrogen, sulphonato or C₁₋₄-acyl, and R^{Z} is hydrogen, C₁₋₄alkyl or C₁₋₄acyl; and
most preferably R⁸ sulphonato or a group of formula III wherein R^{Z} is acetyl or hydrogen, R^{Y} is hydrogen, and R^{X} is hydrogen or methyl;
preferably R⁹ is hydrogen, α-L-fucopyranosyl or arabinosyl, and more preferably R⁹ is hydrogen;
preferably R¹⁰ is hydrogen, methyl or hydroxymethyl, and more preferably R¹⁰ is methyl;
preferably R¹¹ is hydrogen, mannopyranosyl, glycerol or C₁₋₄alkyl, and more preferably, R¹¹ is hydrogen;
preferably R¹² is hydrogen or C₁₋₄acyl, and more preferably R¹² is hydrogen;
preferably m is 1; and
preferably n is 2.

In one embodiment the invention provides a method of modulating angiogenesis in a mammal comprising administering to the mammal a therapeutically effective amount of a oligosaccharide formula I, wherein R¹ is —X-Alk and wherein X is —C(O)— and Alk is selected from an optionally substituted, straight chain or branched, alkyl, alkenyl or alkynyl group having from 2 to 30 carbon atoms.

In another embodiment the invention provides a method of modulating angiogenesis in a mammal comprising administering to the mammal a therapeutically effective amount of an oligosaccharide of formula I or a pharmaceutically acceptable salt thereof, wherein R⁸ is hydrogen, sulphonato, C₁₋₄acyl, an unsubstituted monosaccharide, or a substituted monosaccharide of formula III:

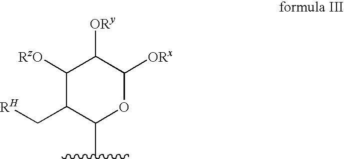

wherein:
R^{x} is hydrogen, C₁₋₄alkyl or C₁₋₄acyl, R^{y} is hydrogen, sulphonato or C₁₋₄acyl, R^{z} is hydrogen, C₁₋₄-alkyl or C₁₋₄acyl, and R^{H} is hydrogen or OR^{P}, wherein R^{P} is hydrogen, C₁₋₄alkyl or C₁₋₄acyl.

In a further embodiment, the invention provides methods of modulating angiogenesis in a mammal, comprising administering to the mammal a therapeutically effective amount of a oligosaccharide of formula I, wherein:

R¹ is hydrogen, —X-Alk or —X-Alk¹-Q-Y-Alk²;
R² is hydrogen or C₁₋₄alkyl;
R³, R⁴ and R⁵ are independently selected from hydrogen, carbamoyl and C₁₋₄acyl;
R⁶ is hydrogen, C₁₋₄acyl or α-L-fucopyranosyl;
R⁷ is independently selected from an acetamide or a hydroxyl group;
R⁸ is hydrogen, arabinosyl, sulphonato, C₁₋₄acyl or a substituted monosaccharide of formula IV:

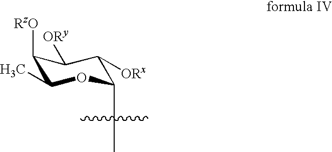

wherein:
R^{x} is hydrogen, C₁₋₄alkyl or C₁₋₄acyl, R^{y} is hydrogen, sulphonato or C₁₋₄acyl, and R^{z} is hydrogen, C₁₋₄alkyl or C₁₋₄acyl;
R⁹ is hydrogen, α-L-fucopyranosyl or arabinosyl;
R¹⁰ is hydrogen, or optionally substituted methyl;
R¹¹ is hydrogen, mannosyl, glycerol or C₁₋₄alkyl;
R¹² is hydrogen or C₁₋₄acyl;
m is 1; and n is 1 or 2.

In yet a further embodiment the invention provides a method of modulating angiogenesis in a mammal comprising administering to the mammal a therapeutically effective amount of an oligosaccharide of formula V or a pharmaceutically acceptable salt thereof:

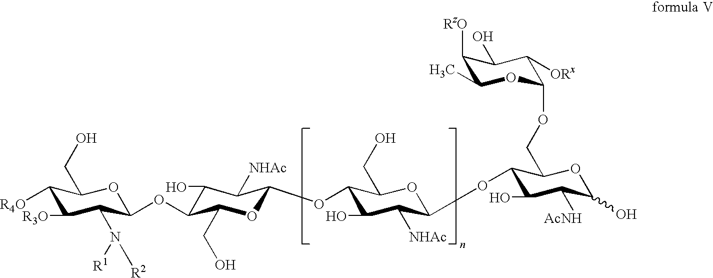

wherein:
R¹ is hydrogen, —X-Alk or —X-Alk¹-Q-Y-Alk²:
R² is hydrogen or methyl;
R³ and R⁴ are independently selected from hydrogen and carbamoyl;
R^{z} is hydrogen or acetyl;
R^{x} is hydrogen or methyl; and
n is 1 or 2.

In a still further embodiment the invention provides a method of modulating angiogenesis in a mammal comprising administering to the mammal a therapeutically effective amount of an oligosaccharide of formula V or a pharmaceutically acceptable salt thereof wherein: R¹ is selected from —X-Alk or —X-Alk¹-Q-Y-Alk²; R², R³ and R⁴ are each hydrogen, R^{z} is hydrogen or acetyl, and Rx is hydrogen or methyl.

In a still further embodiment the invention provides a method of modulating angiogenesis in a mammal comprising administering to the mammal a therapeutically effective amount of a oligosaccharide of formula V or a pharmaceutically acceptable salt thereof wherein: R¹, R², R³ and R⁴ are each hydrogen, R^{z} is hydrogen or acetyl, and Rx is hydrogen or methyl.

In a still further embodiment the invention provides a method of modulating angiogenesis in a mammal comprising administering to the mammal a therapeutically effective amount of an oligosaccharide of formula V or a pharmaceutically acceptable salt thereof wherein: R¹ is selected from —X-Alk or —X-Alk¹-Q-Y-Alk², R² is hydrogen or methyl, R³ and R⁴ are each carbamoyl, R^{Z} is hydrogen or acetyl, and R^{X} is hydrogen or methyl.

In yet a further embodiment the invention provides a method of modulating angiogenesis in a mammal comprising administering to the mammal a therapeutically effective amount of an oligosaccharide of formula VI or a pharmaceutically acceptable salt thereof:

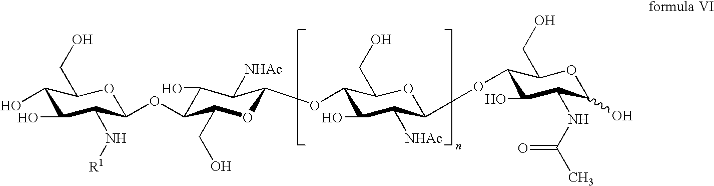

wherein:
R¹ is hydrogen, —X-Alk or —X-Alk¹-Q-Y-Alk²;
n is 1 or 2.

In yet another embodiment the invention provides a method of modulating angiogenesis in a mammal comprising administering to the mammal a therapeutically effective amount of an oligosaccharide of formula VII or a pharmaceutically acceptable salt thereof:

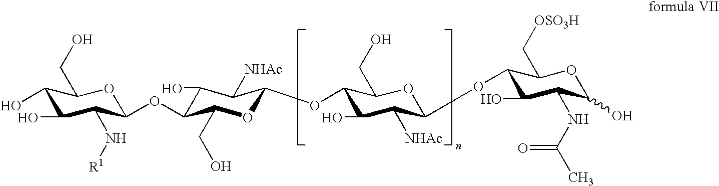

wherein:
R¹ is hydrogen, —X-Alk or —X-Alk¹-Q-Y-Alk²;
n is 1 or 2.

In still another embodiment the invention provides a method of modulating angiogenesis in a mammal comprising administering to the mammal a therapeutically effective amount of an oligosaccharide of formula VIII or a pharmaceutically acceptable salt thereof:

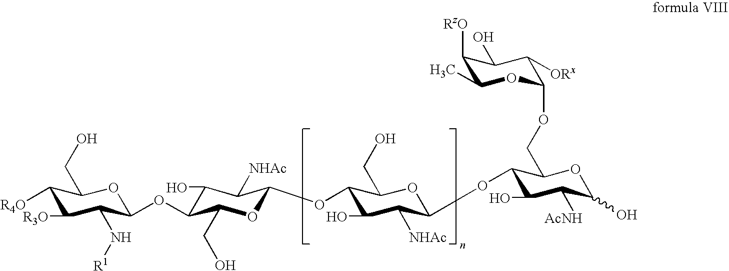

wherein:

### R¹ is —X-Alk¹-Q-Y-Alk²;

wherein:
X is selected from —C(O)—, —C(NR^{N})—, —C(S)—, —SO₂—, —P(O)(OR^{N})— wherein R^{N} is hydrogen, hydroxy, amino, optionally substituted C₁₋₈alkyl, optionally substituted C₂₋₈alkenyl, optionally substituted C₂₋₈alkynyl, optionally substituted C₁₋₄alkylaryl, and optionally substituted aryl;
Alk¹ is absent or present and is selected from an optionally substituted divalent C₁₋₁₀alkyl, optionally substituted divalent C₂₋₁₀alkenyl and optionally substituted divalent C₂₋₁₀alkynyl chain;
Q is absent or present and is selected from an optionally substituted divalent cycloalkyl, optionally substituted divalent cycloalkenyl, optionally substituted divalent heterocycle, optionally substituted divalent aryl or optionally substituted divalent heteroaryl system;
Y is absent or present and is selected from —NH—, —O—, —S—, —NHC(O)—, —C(O)NH—, NHSO₃—, —C(R^{G})═N—N—, —NHC(O)NH—, —NHC(S)NH—, —NHC(NH)NH—, —C(R^{G})═N— and —N═C(R^{G})—, wherein R^{G} is hydrogen, optionally substituted C₁₋₆alkyl, optionally substituted arylC₁₋₄alkyl, optionally substituted aryl or optionally substituted heteroaryl; and
Alk² is absent or present and is selected an optionally substituted, straight chain or branched, alkyl, alkenyl or alkynyl group having from 1 to 30 carbon atoms;
   R^{Z} is hydrogen or acetyl;
   R^{X} is hydrogen or methyl; and
   n is 1 or 2.

It is preferred that the Nod factor or derivative thereof used in accordance with the invention is neutral, or does not have a charge, positive or negative, of greater magnitude than 1.

In another embodiment the invention provides methods of preventing or treating an angiogenesis associated disorder in a mammal comprising administering to the mammal a therapeutically effective amount of a Nod factor or derivative thereof.

Generally, the invention provides methods of preventing or treating disorders in mammals through modulation of angiogenesis. Accordingly, the invention provides a method of preventing or treating disorders in mammals through inhibiting angiogenesis with a Nod factor or derivative thereof.

Disorders that may be treated by inhibiting angiogenesis include, but are not limited to, all types of cancer, chronic inflammatory diseases and ocular neovascular disease as well as obesity. Cancer treatment involves inhibiting primary tumour formation and metastasis in solid tumours such as rhabdomyosarcomas, retinoblastoma, Ewing sarcoma, neuroblastoma, osteosarcoma, colon, prostate, head and neck, breast, bladder, liver, pancreatic, lung, CNS, Paget's disease and blood-born tumours such as leukemia as well as benign tumours such as hemangioma. Chronic inflammatory diseases include rheumatoid arthritis, ulcerative colitis, Crolin's disease, systemic lupus erythematosis, multiple sclerosis, psoriasis, sarcoid/sarcoidosis and Behcet's disease. Ocular diseases include diabetic retinopathy, chronic uveitis/vitritis, retinopathy of prematurity, Eale's disease, infections causing a retinitis or choroiditis, presumed ocular histoplasmosis, trauma and post-laser complications, as well as, but not limited to, diseases associated with rubeosis (neovascularisation of the angle) and diseases caused by the abnormal proliferation of fibrovascular or fibrous tissue including all forms of proliferative vitreoretinopathy.

It is envisioned that the compounds of the inventions can be combined with other drugs to form combination therapeutics, for example, when treating a cancer related disorder, the compounds of the invention may be combined with at least one additional anti-cancer, anti-metastatic or anti-neoplastic agent.

The present invention is associated with the treatment of disorders in mammals through modulation of angiogenesis. In one aspect the treatment is provided by inducing angiogenesis with a Nod factor or derivative thereof. This treatment may be associated with establishing, maintaining or extending vascularisation.

The invention therefore provides a method of preventing or treating an angiogenesis associated disorder in a mammal with a Nod factor or derivative thereof by inducing angiogenesis, wherein the disorder is associated with tissue or organ transplant (including artificial organs), stimulation of collateral circulation, conditions that exhibit insufficient or sub-optimal angiogenesis, tissue infarction, arterial stenosis, coronary heart disease, thromboangitis obliterans, wound healing, ischemia, promoting new blood vessel growth, improving blood flow, and reducing tissue damage.

Methods of treatment of angiogenesis related disorders utilising Nod factors and derivatives thereof may be associated with establishing, maintaining or extending angiogenesis for treatment or prevention of disorders and conditions including, but not limited to: ischemia, including without limitation ischemic stroke (for example, from stenosis), cerebral ischemia, myocardial ischemia (for example, coronary artery disease), intestinal ischemia, retinal or ocular ischemia, spinal ischemia; circulatory disorders; vascular disorders; myocardial disease; pericardial disease; congenital heart disease; peripheral vascular pathologies (associated, for example, with diabetes); infertility due to insufficient endometrial vascularisation; occluded blood vessels, for example, due to atherosclerosis; conditions involving the pathology of endothelial cells, such as endothelial ulcerations in diabetics; peptic ulcerations; or wounds (eg. due to surgery, burns fracture, cuts, or infection).

Methods of treatment of angiogenesis related disorders with Nod factor or derivative thereof may also be associated with establishing, maintaining or extending angiogenesis in tissues, including but not limited to: fibrous, muscle, endothelial, epithelial, vesicular, cardiac, cerebrovascular, vascular tissues, or avascular tissues, including the transparent structures of the eye (eg. corneas, lens, vitreous), discs, ligaments, cartilage, tendons, epidermis etc.; and organs (including artificial organs) for transplantation, including but not limited to heart, liver, lung, kidney, skin, pancreas, eye, and organs in need of regeneration. When the organs are to be transplanted, the compounds, compositions or methods of this invention may be applied to the tissues or organs prior to transplantation (eg. in vitro) or may be administered to the organ transplant recipient (eg. in vivo).

Methods of treatment of angiogenesis related disorders with Nod factor or derivative thereof may also be associated with establishing, maintaining or extending angiogenesis to facilitate better vascularisation and tolerance of an implant or prosthesis, or to inhibit restenosis of stents of artificial implants where the implants include but are not limited to mammary implants, penile implants, artificial urinary sphincters or prostheses.

The compounds of formula I may be produced by biochemical methods. Bacterium containing Nod factors can be cultured in a broth such as yeast extract mannitol broth (YEM) and, at the end of exponential growth phase, spiked with a flavonoid such as genistein. After further incubation, Nod factor oligosaccharides can be harvested by extraction of the media with an alcohol such as n-butanol. After separation of the phases followed rotary evaporation of the organic fraction, the resulting residue is typically redissolved in a solvent such as acetonitrile and purified by reverse-phase chromatography, for example with a C-18 preparative chromatography column. The eluted Nod factor fraction may be further purified by preparative HPLC (Soulemanov, A., et al, Microbiology Research, 2002, 157, 25-28).

In a variation to the above procedure, Nod factors can be isolated solely from the cultured medium according to the methods described in Roche, P., et al., The Journal of Biological Chemistry, 1991, 266(17), 10933-10940. In a further variation of the above procedure, Nod factors can be isolated from membrane lipid extracts of pelleted cells according to the methods described in Orgambide, G., et al, Biochemistry, 1995, 34, 3832-3840.

The compounds of the present invention may also be chemically synthesised using methods of protecting group manipulation including: protection, deprotection and the appropriate selection of protecting groups orthogonal to each other. These methods are analogous to those disclosed in the prior art, for example, description of appropriate protecting groups can be found in “Protection Groups in Organic Synthesis” Theodora W. Greene, Peter G. M. Wuts, 3rd Edition, June 1999, John Wiley & Sons Inc.

It is envisaged that, as required, carbohydrate monosaccharide building blocks can be designed to allow access to a wide rage of selectively derivatised Nod factors by using orthogonal protecting group chemistry.

It is further envisaged that compounds of the present invention may be prepared using methods of chemical synthesis analogous to those described in the prior art. For example, it is proposed that compounds 2 and 3 of the examples could be prepared according to the following series of synthetic conversions which are generally known to the art of carbohydrate chemistry. A monosaccharide donor 8 protected with a temporary protecting group (T¹) and derivatised with a leaving group (L¹) could potentially be reacted with an orthogonally protected acceptor 9, wherein the temporary protecting groups T² and T³ of acceptor 9 are orthogonal to T¹ of donor 8. The permanent protecting group (P¹) of acceptor 9 should be orthogonal to all conditions used to cleave temporary protecting groups and the group NP^{N} should be a permanent nitrogen-protecting group.

In an exemplary proposed procedure, donor 8, wherein L¹ is a thiophenyl group and T¹ are acetyl groups, is reacted in the presence of an activating agent such as NIS TfOH with an acceptor 9, wherein T² is a t-butyldiphenylsilyl group, T³ is a 4-methoxybenzyl group, P¹ is a benzyl group and NP^{N} is phthalimido group, to form a β(1→4)-linked disaccharide 10. The formation of analogous disaccharides has been described in the prior art, for example, Robina, I., et al, Tetrahedron, 2002, 58, 512-530. In a general sense, a disaccharide such as 10 may then be sequentially subjected to the standard protecting group manipulations in order to cleave the T¹ groups to afford the selectively derivatised disaccharide 11. For example, if T¹ were acetyl groups, then derivative 10 could be sequentially subjected to Zemplen conditions, benzylidene ring formation, benzylation followed by selective ring opening to afford an exemplary orthogonally protected disaccharide acceptor 11, wherein P¹, NP^{N}, T² and T³ are as mentioned above. A selectively protected disaccharide 11 could be then glycosylated by a selectively derivatised trisaccharide donor 12 (the synthesis of which is discussed in Scheme 2 below). An exemplary trisaccharide 12 could have L² as a trichloroacetimidate leaving group and NP^{N1} as an azide protecting group.

The significance of the two different amino protecting groups NP^{N} and NP^{N1} is that typically the non-reducing end glucosaminyl residue of a Nod factor is derivatised with a different 2-deoxy-2-amino functional group than the remaining 2-deoxy-2-amino functional groups of the Nod factor. For example, the terminal non-reducing 2-deoxy-2-amino group is typically a saturated or unsaturated fatty acid, which may or may not be N-alkylated, whilst the remainder of the 2-deoxy-2-amino functional groups of a Nod factor are typically, although not always, acetamido groups. Thus, the use of two different, and orthogonal, amino protecting groups, should allow for selective derivatisation of the non-reducing glucosaminyl terminus of a Nod factor.

Thus, a trichloroacetimidate donor 12, as mentioned above, may be activated in the presence of a promoter, such as TMSOTF, and a suitably protected acceptor 11, to form a β(1→4)-linked pentamer. The pentamer may be further selectively derivatised, for example, if NP^{N} where phthalimido protected functions, reaction with hydrazine hydrate in alcohol under heat, followed by acetylation, for example, with acetic anhydride, would allow the formation of a pentasaccharide such as 13.

Many Nod factors have selective functionalisation at the 6-position of the reducing end glucosaminyl residue, such as a fucose, arabinose, acetyl or sulphate moiety as well as the standard hydroxyl group. The use of a temporary protecting group T³, orthogonal to both T¹ and T², should allow for selective derivatisation in this position if required. For example, T³ could be a p-methoxybenzyl protecting group, which can be selectively removed under neutral oxidative conditions, for example with ceric ammonium nitrate or DDQ or, alternatively, under acidic conditions for example with TFA. The resulting primary hydroxyl group can be then be derivatised, for example, by glycosylation with a fucopyranosyl donor to afford a hexasaccharide such as 15. Suitable fucopyranosyl donors, analogous to those employed in the schemes of the invention have been described in the prior art, for example: Akira Hasegawa, et al, Carbohydrate Research, 1995, 274, 155-163; and Debenham, J. S., et al, J. Org. Chem., 1996, 61, 6478-6479.

At this stage of the synthesis, it is proposed that the orthogonal amine protecting group NP^{N1} could be removed and reacted with a suitable activated fatty acid group. For example, if NP^{N1} of hexasaccharide 15 were an azide function, it could be selectively reduced, for example, with activated zinc in the presence of ammonium chloride, and then acylated with an appropriate fatty acid to form a protected lipo-chitooligosaccharide.

Alternatively, if an amino derivative is desired, such as compounds 6 and 7 of the examples, then the derivatised free amine is not reacted further. The remaining steps to generate the final product require the removal of all remaining temporary and permanent protecting groups. For example, if T² were a t-butyldiphenylsilyl group, it could be selectively removed by treatment with a fluoride ion source such as tert-butylammonium fluoride (TBAF). If P¹ were benzyl groups, they could be removed in the final step by hydrogenolysis to afford deprotected lipochitooligosaccharides 16.

When R² is a hydrogen atom and R¹ is a C_{18:1} fatty acid, compound 16 of Scheme 1 describes compound 3 of the examples. When R² is a methyl group and R¹ is a C_{18:1} fatty acid, compound 16 of Scheme 1 describes compound 3 of the examples.

When R¹ and R² are hydrogen atoms, compound 14 of Scheme 1 describes compound 6 of the examples. When R¹ is a hydrogen atom and R² is a methyl group, compound 14 of Scheme 1 describes compound 7 of the examples.

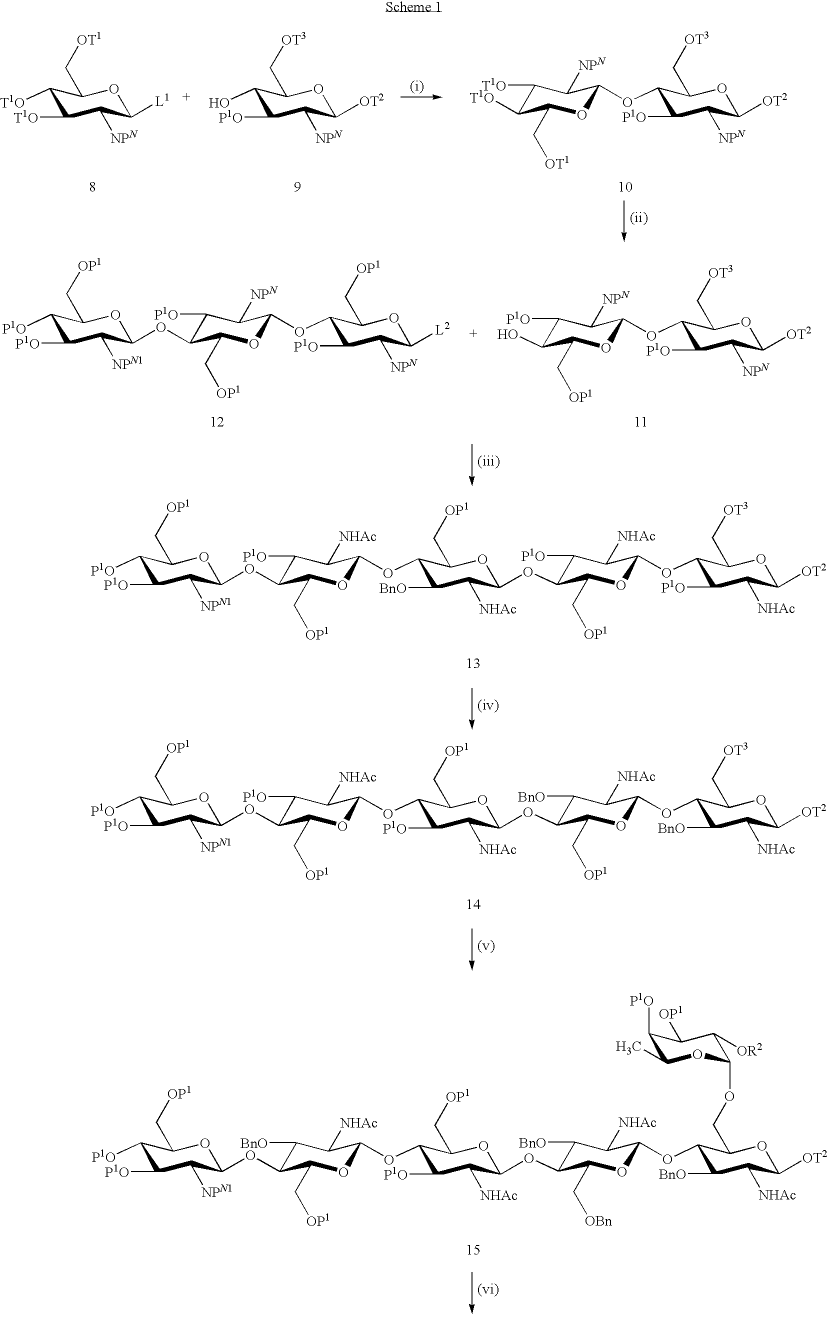

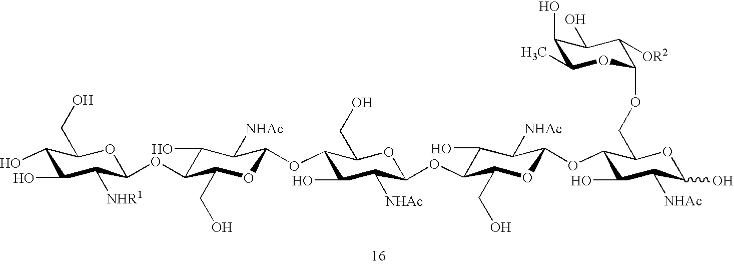

It is envisioned that trisaccharides 12 from Scheme 1 can be prepared by the methodology shown in Scheme 2. A donor sugar, for example, an azido protected, tris-benzyl trichloroacetimidate (TCA) donor sugar (L¹=TCA, P¹=Bn, and NP^{N1}=N₃) can be potentially reacted with a disaccharide acceptor 18 in the presence of a promoter such as TMSOTf, to afford trisaccharide 19. Methods of preparation of disaccharides, such as acceptor 18, have been described in the prior art, for example, Robina, I., et al, Tetrahedron, 2002, 58, 512-530. It is envisaged that the anomeric ratio resulting from the reaction of a protected monosaccharide 17 with a disaccharide 18 could be influenced through variation of temperature and choice of solvent in order to drive the predominant formation of a beta anomer. Anomeric mixtures of protected oligosaccharides can be purified by methods known to the art, such as crystallisation and chromatographic purification. The temporary protecting group T³ is removed and the resulting hydroxyl group converted to a leaving group L². For example, if T¹ was an anomeric p-methoxy benzyl ether protecting group, it could be removed using conditions similar to those previously described above, to afford a lactol which could be subsequently reacted with trichloroacetonitrile in the presence of a base, such as potassium carbonate or DBU, to form a TCA trisaccharide donor 12.

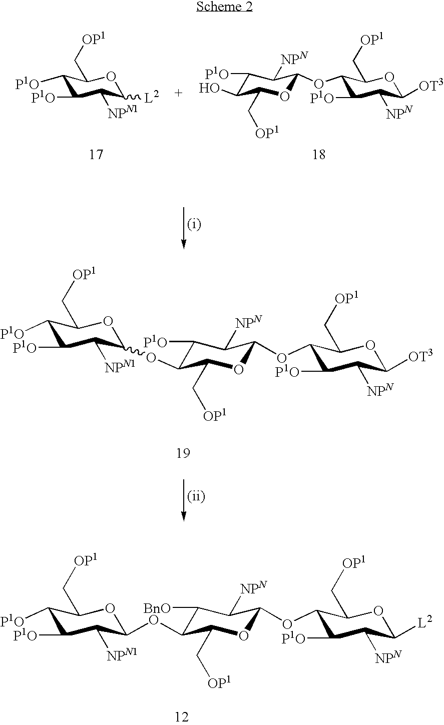

It is envisaged that the compounds of the invention can be prepared by recombinant enzyme technology. For example recombinant Nod factor glycosyltransferases could be used to synthesise the oligomeric glucosaminyl backbones: Samain, E., et. al., Carbohydrate Research, 1997, 302, 235-242; Kamst, E., et. al., Carbohydrate Research, 1999, 321, 176-189; Samain, E., et. al., J. Biotechnol., 1999, 72, 33-47; Dumon, C., et. al., Biotechnol, Prog., 2004, 20(2), 412-419; and Ramussen, M. O., et. al., Org. Biomol. Chem., 2004, 2, 1908-1910. It is envisioned that α(1→2)-, α(1→3)- and α(1>6)-fucosylation (e.g. alpha-1,6-fucosyltransferase from A. caulinodans nodZ gene) could be also achieved using similar recombinant technology, for example, methods for enzymatic fucosylation can be found in the prior art document WO 01/23398.

It is envisaged that lipidic and aromatic side chains of Nod factors of formula I may also be prepared by methods analogous to those disclosed in the prior art (see Ghomsi, J-N., T., Tetrahedron Letters, 2005, 46, 1537-1539). Further, fully unprotected Nod factor oligosaccharides that are free amines, ie. 2-deoxy-2-amino functionalised at the non-reducing termini, may be selectively N-acylated with organic acids, as a result of the difference in reactivity between amino and hydroxyl functions, to provide the corresponding lipo-chitooligosaccharides. Any suitable organic acid such as, for example, optionally substituted benzoic acids; optionally substituted 2-phenyl-acetic acids; optionally substituted 3-phenyl-propionic acids; optionally substituted, saturated or unsaturated fatty acids, or sulpho- or phospho-lipids. The organic acids may be activated by conversion, for example, to the acid chloride form or by conversion to a carbodiimide intermediate in situ.

In addition to the pentamers and hexamers described above, compounds of formula I of the invention, wherein m+n=2, can be prepared by methods analogous to those disclosed in Robina, I., et al, Tetrahedron, 2002, 58, 512-530, and further, compounds of formula I wherein n=1, and in which the reducing glucosaminyl moiety is fucosylated may be prepared by methods analogous to those disclosed in Shinji Ikeshita et al, Carbohydrate Research, 1995, 266, C₁-C₆. The syntheses of reducing end 6-O-suphonato-tetramer Nod factors and derivatives thereof, which are Nod factors of formula I, are disclosed in Grenouillat, N., et. al., Angew. Chem. Int. Ed., 2004, 43, 4644-4646. Similarly, the synthesis of NodRm-IV factors, which are Nod factors of formula I, are described in Nicolaou, K. C., et al., J. Am. Chem. Soc., 1992, 114, 8701-8702.

The Nod factor or derivative thereof of formula I may be characterised by methods analogous known to the art, for example, the Nod factor or derivative thereof of formula I may be identified by mass spectroscopy (Prome, J., C., et al, International Journal of Mass Spectroscopy, 2002, 219, 703-716). Alternatively, Nod factor or derivative thereof of formula I may be structurally analysed by degradation studies in conjunction with mass spectroscopy analysis (Soria-Diaz, M. E., et al, Carbohydrate Research, 2003, 338, 237-250; Gil-Serrano, A. M., et al., Carbohydrate Research, 1997, 303, 435-443). Additionally, functional side-chains of Nod factor or derivative thereof of formula I may be characterised by methods analogous known to the art (Treilhou, M., et al, Journal of the American Society for Mass Spectroscopy, 2000, 11, 301-311).

Other analogous methods for the preparation, isolation, purification and characterisation of Nod factors of formula I can be found in U.S. Pat. No. 5,449,717 and U.S. Pat. No. 5,646,018.

Where appropriate, the salts of the compound of formula I are preferably pharmaceutically acceptable, but it will be appreciated that non-pharmaceutically acceptable salts are also useful according to the present invention, since these are useful as intermediates in the preparation of pharmaceutically acceptable salts. The pharmaceutically acceptable salts may include conventional non-toxic salts or quartenary ammonium salts of these compounds, which may be formed, eg. from organic or inorganic acids or bases. Examples of such acid addition salts include, but are not limited to, those formed with pharmaceutically acceptable acids such as acetic, propionic, citric, lactic, methanesulphonic, toluenesulphonic, benzenesulphonic, salicyclic, ascorbic, hydrochloric, orthophosphoric, sulphuric and hydrobromic acids. Base salts include, but are not limited to, those formed with pharmaceutically acceptable cations, such as sodium, potassium, lithium, calcium, magnesium, ammonium and alkylammonium. Also, basic nitrogen-containing groups may be quaternised with such agents as lower alkyl halides, such as methyl, ethyl, propyl, and butyl chlorides, bromides and iodides; dialkyl sulfates like dimethyl and diethyl sulfate, and others.

The compounds of the invention may be in crystalline form or as solvates (eg. hydrates) and it is intended that both forms are within the scope of the present invention. Methods of solvation are generally known within the art.

Pharmaceutically acceptable derivatives may include any pharmaceutically acceptable hydrate or any other compound or pro-drug which, upon administration to a subject, is capable of providing (directly or indirectly) a compound of formula I or a desirably active metabolite or residue thereof.

Any compound that is a pro-drug of a compound of formula I is within the scope and spirit of the invention. The term “pro-drug” is used in its broadest sense and encompasses those derivatives that are converted in vivo to the compounds of the invention. Such derivatives would readily occur to those skilled in the art and include, for example, compounds where a free hydroxy group is converted into an ester derivative. Examples of ester derivatives include alkyl esters and phosphate esters.

It will be appreciated that derivatives of the compound of formula I have asymmetric centres and therefore are capable of existing in more than one stereoisomeric form. The invention extends to each of these forms individually and to mixtures thereof, including racemates. The isomers may be separated conventionally by chromatographic methods or using a resolving agent. Alternatively, the individual isomers may be prepared by asymmetric synthesis using chiral intermediates.

The invention also provides the use of a compound of formula I or a pharmaceutically acceptable salt thereof in the manufacture of a pharmaceutical composition for the treatment of a disease state or condition, where to a certain extent modulation (e.g. inhibition) of angiogenesis is desirable. Accordingly the invention provides an angiogenesis modulating pharmaceutical composition comprising a Nod factor or derivative thereof, and further provides for the use of a Nod factor or derivative thereof in the modulation of angiogenesis.

The pharmaceutical compositions can be used in the treatment of a variety of diseases mediated by angiogenesis. Disorders that may be treated by inhibiting angiogenesis include, but are not limited to, all types of cancer, chronic inflammatory diseases and ocular neovascular disease as well as obesity. Cancer treatment involves inhibiting primary tumour formation and metastasis in solid tumours such as rhabdomyosarcomas, retinoblastoma, Ewing sarcoma, neuroblastoma, osteosarcoma, colon, prostate, head and neck, breast, bladder, liver, pancreatic, lung, CNS, Paget's disease and blood-born tumours such as leukemia as well as benign tumours such as hemangioma. Chronic inflammatory diseases include rheumatoid arthritis, ulcerative colitis, Crohn's disease, systemic lupus erythematosis, multiple sclerosis, psoriasis, sarcoid/sarcoidosis and Behcet's disease. Ocular diseases include diabetic retinopathy, chronic uveitis/vitritis, retinopathy of prematurity, Eale's disease, infections causing a retinitis or choroiditis, presumed ocular histoplasmosis, trauma and post-laser complications, as well as, but not limited to, diseases associated with rubeosis (neovascularisation of the angle) and diseases caused by the abnormal proliferation of fibrovascular or fibrous tissue including all forms of proliferative vitreoretinopathy.

In prophylactic applications, pharmaceutical compositions or medicaments of Nod factor or derivative thereof are administered to a patient susceptible to, or otherwise at risk of, a disease or condition related to angiogenesis (e.g. a neoplastic or metastatic disease) in an amount sufficient to eliminate or reduce the risk, lessen the severity, or delay the onset of the disease, including biochemical, histologic and/or behavioural symptoms of the disease, its complications and intermediate pathological phenotypes presenting during development of the disease.

In therapeutic applications, compositions or medicaments are administered to a patient suspected of, or already suffering from, such a disease in an amount sufficient to cure, or at least partially arrest, the symptoms of the disease (biochemical, histologic and/or behavioural), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient prophylactic or therapeutic response has been achieved. Typically, the prophylactic or therapeutic response is monitored and repeated dosages are given if the response starts to wane.

While it is possible that, for use in therapy, a compound of the invention may be administered as the neat chemical, it is preferable to present the active ingredient as a pharmaceutical formulation.

The invention thus further provides pharmaceutical formulations comprising a compound of the invention or a pharmaceutically acceptable salt or derivative thereof together with one or more pharmaceutically acceptable carriers therefor and, optionally, other therapeutic and/or prophylactic ingredients. The carrier(s) must be acceptable in the sense of being compatible with the other ingredients of the formulation and not deleterious to the recipient thereof.

The anti-angiogenic treatment defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. For example, in the field of medical oncology it is normal practice to use a combination of different forms of treatment to treat each patient with cancer. In medical oncology, the other component(s) of such conjoint treatment in addition to the anti-angiogenic treatment defined hereinbefore may be surgery, radiotherapy or chemotherapy. Such chemotherapy may cover three main categories of therapeutic agent: (i) other anti-angiogenic agents such as those which inhibit the effects of vascular endothelial growth factor (for example, the anti-vascular endothelial cell growth factor antibody avastin) and those that work by different mechanisms from those defined hereinbefore (for example, PI-88, linomide, inhibitors of integrin AVP3 function, angiostatin, razoxin) and including vascular targeting agents (for example, combretastatin phosphate and N-acetylcolchinol-O-phosphate); (ii) cytostatic agents such as antioestrogens (for example, tamoxifen, toremifene, raloxifene, droloxifene, iodoxyfene), oestrogen receptor down regulators (for example, fulvestrant), progestogens (for example, megestrol acetate), aromatase inhibitors (for example, anastrozole, letrazole, vorazole, exemestane), antiprogestogens, antiandrogens (for example, flutamide, nilutamide, bicalutamide, cyprotelone acetate), luteinising hormone-releasing hormone (LHRH) agonists and antagonists (for example, goserelin acetate, luprolide, buserelin), inhibitors of 5a-reductase (for example, finasteride), anti-invasion agents (for example, metalloproteinase inhibitors like marimastat and inhibitors of urokinase plasminogen activator receptor function) and inhibitors of growth factor function (such growth factors include, for example, platelet derived growth factor and hepatocyte growth factor), such inhibitors include growth factor antibodies, growth factor receptor antibodies, (for example, the anti-erbb2 antibody trastuzumab and the anti-ERBBL antibody Erbitux), farnesyl transferase inhibitors, tyrosine kinase inhibitors, for example, inhibitors of the epidermal growth factor family (i.e., EGFR family tyrosine kinase inhibitors such as N-(3-chloro-4-fluorophenyl)-7-methoxy-6-(3-morpholinopropoxy)quinazolin-4-amine (gefitinib), N-(3-ethynylphenyl)-6,7-bis(2-methoxyethoxy)quinazolin-4-amine (erlotinib) and 6-acrylamido-N-(3-chloro-4-fluorophenyl)-7-(3-morpholinopropoxy)quinazolin-4-amine and serine/threonine kinase inhibitors); and (iii) antiproliferative/antineoplastic drugs and combinations thereof, as used in medical oncology, such as antimetabolites (for example, antifolates such as methotrexate, fluoropyrimidines such as 5-fluorouracil, tegafur, purine and adenosine analogues, cytosine arabinoside); antitumour antibiotics (for example, anthracyclines such as adriamycin, bleomycin, doxorubicin, daunomycin, epirubicin and idarubicin, mitomycin-C, dactinomycin, mithramycin); platinum derivatives (for example, cisplatin, carboplatin); alkylating agents (for example, nitrogen mustard, melphalan, chlorambucil, busulphan, cyclophosphamide, ifosfamide, nitrosoureas, thiotepa); antimitotic agents (for example, vinca alkaloids like vincristine, vinblastine, vindesine, vinorelbine, and taxoids like taxol, taxotere); topoisomerase inhibitors (for example, epipodophyllotoxins like etoposide and teniposide, amsacrine, topotecan, camptothecin and also irinotecan); also enzymes (for example, asparaginase); and thymidylate synthase inhibitors (for example, raltitrexed and histone deacetylase inhibitors); and additional types of chemotherapeutic agent include: (iv) biological response modifiers (for example, interferon); (v) antibodies (for example, edrecolomab); (vi) antisense therapies, for example, those which are directed to the targets listed above, such as ISIS 2503, an anti-ras antisense; (vii) gene therapy approaches, including, for example, approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and (viii) immunotherapy approaches, including, for example, ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies. In addition, the presently described compound may be used in combination with other forms of cancer therapy (eg. radiation therapy).

The invention also provides the use of a compound of formula I in the manufacture of a medicament for the treatment of a disease state or condition, where to a certain extent induction or maintenance of angiogenesis is desirable. For example, promoting new blood vessel growth, improving blood flow, or reducing tissue damage. Such disorders or conditions may include, for example, those conditions that exhibit insufficient or sub-optimal angiogenesis.

Thus, the compounds, compositions or methods of this invention may be used for treatment or prevention of disorders and conditions such as ischemia including, without limitation, ischemic stroke (for example, from stenosis), cerebral ischemia, myocardial ischemia (for example, coronary artery disease), intestinal ischemia, retinal or ocular ischemia, spinal ischemia; circulatory disorders; vascular disorders; myocardial disease; pericardial disease; congenital heart disease; peripheral vascular pathologies (associated, for example, with diabetes); infertility due to insufficient endometrial vascularisation; occluded blood vessels, for example, due to atherosclerosis; conditions involving the pathology of endothelial cells, such as endothelial ulcerations in diabetics, peptic ulcerations, or wounds (eg. due to surgery, burns fracture, cuts, or infection).

The compounds, compositions, or methods of this invention may be used to promote angiogenesis in, for example, tissues such as fibrous, muscle, endothelial, epithelial, vesicular, cardiac, cerebrovascular, vascular tissues, or avascular tissues, including the transparent structures of the eye (eg. cornea, lens, vitreous), discs, ligaments, cartilage, tendons, epidermis etc.; organs, for example, organs for transplantation or artificial organs (eg. heart, liver, lung, kidney, skin, pancreas, eye), or organs in need of regeneration. For tissue or organ transplants, the compounds, compositions or methods of this invention may be applied to the tissues or organs prior to transplantation (eg. in vitro) or may be administered to the organ transplant recipient (eg. in vivo). The compounds, compositions, or methods of this invention may be used to promote angiogenesis when using artificial implants, for example, mammary implants, penile implants, artificial urinary sphincters, or using prostheses, to facilitate better vascularisation and tolerance of the implant or prosthesis, or to inhibit restenosis of stents.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation or insufflation. The compounds of the invention, together with a conventional adjuvant, carrier or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to two hundred (200) milligrams, per tablet, are accordingly suitable representative unit dosage forms. The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilisers, lubricants, suspending agents, binders, preservatives, tablet disintegrating agents, or an encapsulating material.

In powders, the carrier is a finely divided solid which is in a mixture with the finely divided active component. In tablets, the active component is mixed with the carrier having the necessary binding capacity in suitable proportions and compacted in the shape and size desired.

The powders and tablets preferably contain from 5% or 10% to about 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized moulds, allowed to cool, and thereby to solidify.

Formulations suitable for vaginal administration may be presented as pessaries, tampons, creams, gels, pastes, foams or sprays containing, in addition to the active ingredient, such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The compounds according to the present invention may thus be formulated for parenteral administration (eg. by injection, for example, bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, eg. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilising agents, and the like.

For topical administration to the epidermis, the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents or colouring agents.

Solutions or suspensions are applied directly to the nasal cavity by conventional means, for example, with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved, for example, by means of a metering atomising spray pump. To improve nasal delivery and retention, the compounds according to the invention may be encapsulated with cyclodextrins or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC), for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively, the active ingredients may be provided in the form of a dry powder, for example, a powder mix of the compound in a suitable powder base such as lactose, starch, starch derivatives such as hydroxypropylmethyl cellulose and polyvinylpyrrolidone (PVP).

Conveniently, the powder carrier will form a gel in the nasal cavity. The powder composition may be presented in unit dose form, for example, in capsules or cartridges of, eg. gelatin, or blister packs from which the powder may be administered by means of an inhaler.

In formulations intended for administration to the respiratory tract, including intranasal formulations, the compound will generally have a small particle size, for example, of the order of 1 to 10 microns or less. Such a particle size may be obtained by means known in the art, for example, by micronisation.

When desired, formulations adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Liquids or powders for intranasal administration, tablets or capsules for oral administration and liquids for intravenous or parenteral administration, are preferred compositions.

### BRIEF DESCRIPTION OF THE DRAWINGS

FIG. 1 contains photographic representations illustrating changes in blood vessel morphology in human umbilical vein endothelial cells (HUVEC) after treatment with P1-88, compound 2 and compound 3. Control results are also shown.

The invention will now be described with reference to the following examples that illustrate some preferred aspects of the present invention. However, it is to be understood that the particularity of the following description of the invention is not to supersede the generality of the preceding description of the invention.

### EXAMPLES

Compounds 1 to 3, 6, 7, and 21 to 26 as set out below were supplied by Dr Eric Samain of CERMAV-CNRS, Grenoble, France. Compound 4, and compound 5 (a mixture of NodNRG-V factors from the Rhizobium strain NRG234) were supplied by Prof. William J. Broughton (currently director of the Botany and Plant Biology Department, University of Geneva).

Compound 1 has been described in the following publications: Samain, E., et al., Carbohydrate Research, 1997, 302, 35-42; Gressent, F., et al., Proc. Natl. Acad. Sci. USA, 1999, 96, 4704-4709; and Samain, E., et al., Journal of Biotechnology, 1999, 72, 33-47.

Compound 2 has been described in the following publications: Bec-Ferté, M-P., et al., Biochemistry, 1994, 33, 11782-11788; Gil-Serrano, A. M., et al., Carbohydrate Research, 1997, 303, 435-443; Hungria, M., et al., Soil. Biol. Biochem., 1997, 29(5/6), 819-830; Cohn J, et al., Trends Plant Sci., 1998, 3, 105-110; and D'Haeze, W., et al., Glycobiology, 2002, 12, 79R-10SR (and references therein).

Compound 3 has been described in the following publications: Sanjuan, J., et al., Proc. Natl. Acad. Sci. USA, 1992, 89, 8789-8793; Carlson, R. W., et al., The Journal of Biological Chemistry, 1993, 286(24), 18372-18381; Stokkermans, T. J. W., et al., Plant Physiol., 1995, 108, 1587-1595; Stacey, G., Soil Biol. Biochem., 1995, 27(4/5), 473-483; Cohn, J., et al., Molecular Plant-Microbe Interactions, 1999, 12(9), 766-773; Lian, B., et al., Microbiol. Res., 2002, 157, 157-160; and Soulemanov, A., et al., Microbiol Res., 2002, 157, 25-28.

Compound 4 has been described in the following publications: Price N. P., et al., Mol., Microbiol., 1992, 6(23), 3575-3584; Jabbouri, S., et al., The Journal of Biological Chemistry, 1995, 270(39), 22968-22973; Jabbouri, S., et al., The Journal of Biological Chemistry, 1998, 273(20), 12047-12055.

Compound 5 has been described in U.S. Pat. No. 5,646,018.

Compound 20 has been described in D'Haeze, W., et al., Glycobiology, 2002, 12, 79R-105R (and references therein).

Compound 25 has been described in WO2005063784.

Compounds of Formula

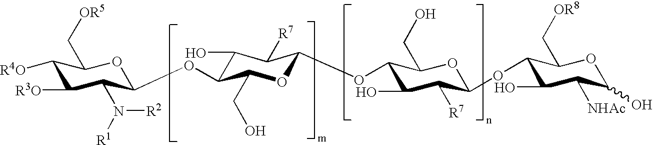

| No. | m | n | R¹ | R² | R³ | R⁴ | R⁵ | R⁷ | R⁸ |
|---|---|---|---|---|---|---|---|---|---|
| 1 | 1 | 2 | H | H | H | H | H | NHAc | H |
| 2 | 1 | 2 | C_{18:1} | H | H | H | H | NHAc | α-L-fucopyranosyl |
| 3 | 1 | 2 | C_{18:1} | H | H | H | H | NHAc | 2-O-methyl-α-L-fucopyranosyl |
| 4 | 1 | 2 | C_{18:1} | H | carbamoyl | carbamoyl | H | NHAc | 4-O-acetyl-2-O-methyl-α-L- |
| | | | | | | | | | fucopyranosyl |
| 5 | 1 | 2 | C_{18:2}/C_{16:0}/C_{18:0}/C_{18:1}/C_{16:1} | Me | carbamoyl/H | carbamoyl/H | carbamoyl/H | NHAc | 3-O-S-2-O-MeFuc; 3-/4-O-Ac- |
| | | | | | | | | | 2-O-MeFuc; 2-O-MeFuc |
| 6 | 1 | 2 | H | H | H | H | H | NHAc | α-L-fucopyranosyl |
| 7 | 1 | 2 | H | H | H | H | H | NHAc | 2-O-methyl-α-L-fucopyranosyl |
| 20 | 1 | 2 | C_{16:2} | H | H | H | H or Ac | NHAc | SO₃H |
| 21 | 1 | 1 | H | H | H | H | H | NHAc | SO₃H |
| 22 | 0 | 0 | H | H | H | H | H | NHAc | H |
| 23 | 1 | 1 | H | H | H | H | H | NHAc | H |
| 24 | 1 | 2 | 2-phenylacetyl | H | H | H | H | NHAc | α-L-fucopyranosyl |
| 25 | 1 | 2 | 3-(undec-4-enyloxy)- | H | H | H | H | NHAc | α-L-fucopyranosyl |
| | | | benzoyl | | | | | | |

| | | | | | | | | |
|---|---|---|---|---|---|---|---|---|
| 26 | 1 | 2 | N(R¹)(R²) = N₃ | H | H | H | NHAc | α-L-fucopyranosyl |

### Example 1

### Rat Aorta Angiogenesis Assay*

Thoracic aortas were excised from three to nine month-old female Fischer rats, rinsed in Hanks balanced salt solution containing 2.5 μg/ml amphotericin B (Sigma, St Louis, Mo.), cleaned of periadventitial fibroadipose tissue and cross-sectioned at 1 mm intervals. The fragments were freed of residual clots. Dissecting and sectioning of the vessels was performed with the aid of a dissecting microscope.

Assays were performed in 48-well culture plates (Costar, Cambridge, Mass.). Five hundred microlitres of 3 mg/ml fibrinogen (bovine plasma, Calbiochem, La Jolla, Calif.) in serum free-Medium 199 (GibcoBRL) was added to each well with 5 μg/ml of aprotinin (Sigma) to prevent fibrinolysis by the vessel fragments. One vessel fragment was placed in the centre of the well and 15 μl of thrombin (50 NIH U/ml in 0.15M NaCl: bovine plasma: Sigma St Louis, Mo.) was added to the well and mixed rapidly with the fibrinogen. Fibrin gel formation usually occurred within 30 seconds and ideally the vessel fragment remained suspended in the centre of the gel. After gel formation, 0.5 ml/well of Medium M199 supplemented with 20% fetal calf serum (FCS) (Sigma), 0.1% 6-aminocaproic acid, 1% L-glutamine, 1%-amphotericin B and 0.6% gentamycin was added. The substances tested for angiogenesis modulating activity (compounds PI-88, 1, 2, 3, 4, 5, 6 and 7) were dissolved in 50% acetonitrile in ultrapure water and diluted at least 1:100 in the supplemented medium M199. Immediately after embedding of vessel fragment in the fibrin gels, 0.5 ml of medium containing the test substance was added to each well and each treatment was performed in six wells. Control cultures received medium without the test substance. Vessels were cultured at 37° C. in 5% CO₂ in air for five days and the medium was changed on day four. Vessel growth was quantified manually under 40× magnification on day five, with growth being estimated as the percentage of the field (×40) around the vessel fragment that was occupied by vessel outgrowths. Results are displayed in Table 1, Table 2, Table 3 and Table 4.
*Brown, K J., Maynes, S F., Bezos, A., Maguire, D J., Ford, M D. & Parish, C R., Laboratory Investigation, 1996, 75, 539-555.

### Example 2

### Mouse Aorta Anglogenesis Assay^{§}

Thoracic aortas were excised from 6-8 week old female C57 BL/6 mice, rinsed in Hanks balanced salt solution containing 2.5 μg/ml amphotericin B (Sigma, St Louis, Mo.) cleaned of periadventitial fibroadipose tissue and cross-sectioned at 1 mm intervals. The fragments were freed of residual clots. Dissecting and sectioning of the vessels was performed with the aid of a dissecting microscope. Assays were performed in 48-well culture plates (Costar, Cambridge, Mass.). Five hundred microlitres of 3 mg/ml fibrinogen (bovine plasma, Calbiochem, La Jolla, Calif.) in serum free-Medium 199 (GibcoBRL)) was added to each well with 5 ug/ml of aprotinin (Sigma) to prevent fibrinolysis by the vessel fragments. One vessel fragment was placed in the centre of the well and 15 ul of thrombin (50 NIH U/ml in 0.15M NaCl: EC 3.4.21.5 bovine plasma: Sigma St Louis, Mo.) was added to the well and mixed rapidly with the fibrinogen. Fibrin gel formation usually occurred within 30 seconds and ideally the vessel fragment remained suspended in the centre of the gel. Immediately after embedding of vessel fragment in the fibrin gels, 0.5 ml/well of Medium M199 supplemented with 20% FCS (Sigma), 0.1% ε-aminocaproic acid, 1% 1-glutamine, 1%-amphotericin B and 0.6% gentamycin was added. The test substance was added to the medium and each treatment was performed in six wells. Control cultures received medium without the test substance. Vessels were cultured at 37° C. in 5% CO₂ in air for 5 days and the medium was changed on day 4. Vessel growth was quantified manually under 40× magnification on day 7, with growth being estimated as the percentage of the field (×40) around the vessel fragment that was occupied by vessel outgrowths (see Table 5).
^{§}Brown, K. J., Maynes, S. F., Bezos, A., Maguire, D. J., Ford, M. D., & Parish, C. R., “Novel In Vitro Assay for Human Angiogenesis”, Laboratory Investigation, 1996, 75, 539-555.

### Example 3

### HUVEC Assay

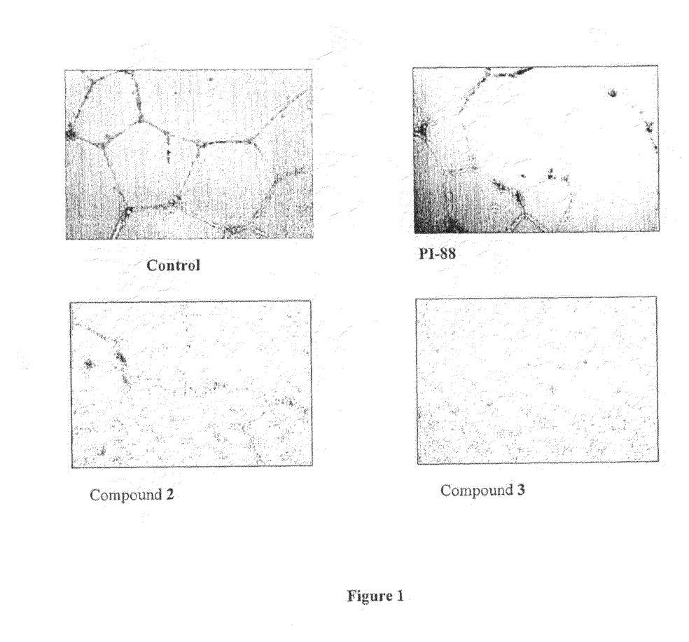

HUVEC (human umbilical vein endothelial cells) form tubes on a matrigel support. The tubes form a “paving tile” formation after overnight incubation. Nod factor and derivative thereof were added at 100 μg/ml to determine if they inhibited tube formation and/or caused a change in tube morphology. PI-88 and all tested compounds affected tube formation (see Table 6 and FIG. 1).

TABLE 1
Rat Aorta Angiogenesis Assay with
Nod factors and Derivatives (1)

| | Conc. | | | |
|---|---|---|---|---|
| Treatment | (μg/mL) | % Growth | % Inhibit. | P value |
| Control† | — | 82 ± 3.2 | — | — |
| PI-88‡ | 100 | 25 ± 5.4 | 69 | <0.0001 |
| 1 | 100 | 53 ± 8.0 | 35 | 0.0007 |
| 1 | 10 | 80 ± 8.4 | 2.4 | NS |
| 2 | 100 | 30 ± 5.5 | 63 | <0.0001 |
| 2 | 10 | 68 ± 10.7 | 17 | NS |
| 3 | 100 | 42 ± 13.9 | 49 | 0.0006 |
| 3 | 10 | 60 ± 6.8 | 27 | 0.0033 |
| 6 | 100 | 53 ± 4.8 | 35 | 0.0003 |
| 6 | 10 | 73 ± 7.5 | 11 | NS |
| 7 | 100 | 90 ± 6.3 | +8 | NS |
| 7 | 10 | 94 ± 4.0 | +12 | NS |
| 20 | 100 μg/ml | 85 ± 3.4 | 2.3 | NS |
| 20 | 10 μg/ml | 88 ± 3.3 | 0 | NS |

TABLE 2
Rat Aorta Angiogenesis Assay with
Nod factors and Derivatives (2)

| | Conc. | | | |
|---|---|---|---|---|
| Treatment | (μg/mL) | % Growth | % Inhibit. | P value |
| Control† | — | 74 ± 3.8 | — | — |
| PI-88‡ | 100 | 39 ± 6.1 | 47 | <0.0001 |
| 4 | 100 | 50 ± 3.7 | 32 | 0.0010 |
| 5 | 100 | 53 ± 3.3 | 28 | 0.0028 |

TABLE 3
Rat Aorta Angiogenesis Assays with Nod factors and Derivatives (3)

| | Testing at 100 μg/mL | | | Testing at 10 μg/mL | | Testing at 1.0 μg/mL | |
|---|---|---|---|---|---|---|---|
| | Assay 1 | Assay 2 | Assay 3 | Assay 4 | Assay 5 | Assay 6 | Assay 7 |
| COMPOUND | % inhibition | % Inhibition | % Inhibition | % Inhibition | % inhibition | % Inhibition | % inhibition |
| CONTROL | N/A | N/A | N/A | N/A | N/A | N/A | N/A |
| PI-88 | 82 | 77 | 68 | 18 | 14 | — | — |
| 2 | 33 | 41 | 49 | 20 | 15 | — | — |
| 2 | — | — | — | 29 | 23 | — | — |
| 21 | 74 | 41 | 40 | 24 | 33 | — | — |
| 22 | 5 | +10 | +6 | 24 | 16 | — | — |
| 23 | — | 66 | 29 | 14 | 24 | — | — |
| 24 | — | +25 | +3 | 3 | 1 | — | — |
| 25 | — | 76 | 73 | 24 | 21 | 8 | 3 |
| 26 | — | 3 | 15 | — | — | — | — |

TABLE 4
Rat aorta angiogenesis Assays with Nod factors and Derivatives (4): % Inhibition days 5, 6, 7

| | Assay 1 | | | ASSAY 2 | | |
|---|---|---|---|---|---|---|
| COMP. | DAY 5 | DAY 6 | DAY 7 | DAY 5 | DAY 6 | DAY 7 |
| | % GROWTH | % GROWTH | % GROWTH | % GROWTH | % GROWTH | % GROWTH |
| CONTROL | 27 | 43 | 68 | 48 | 63 | 83 |
| | % INHIBITION | % INHIBITION | % INHIBITION | % INHIBITION | % INHIBITION | % INHIBITION |
| PI-88 | 91 | 80 | 77 | 66 | 70 | 68 |
| 22 | 33 | +21 | +10 | 6 | 0 | +6 |
| 24 | +67 | +51 | +25 | 6 | 8 | +3 |

### Legend for Tables 1, 2, 3 and 4

^{†} Control is untreated rat aorta
^{‡} PI-88 is a known anti-angiogenic agent and is used as a control.

TABLE 5
Mouse Aorta Angiogenesis

| Compound | Concentration | % Inhibition |
|---|---|---|
| Control | N/A | N/A |
| PI-88 | 100 μg/ml | 93% |
| PI-88 | 10 μg/ml | 30% |
| 2 | 100 μg/ml | 64% |
| 2 | 10 μg/ml | 69% |

TABLE 6
MATRIGEL + HUVEC

| | Inhibition of HUVEC cell “tiling” |
|---|---|
| COMPOUND | and/or change in tube morphology+/− |
| Control | No |
| PI-88 | Yes |
| 1 | Yes |
| 2 | Yes |
| 3 | Yes |
| 6 | Yes |
| 7 | Yes |
| 20 | Yes |

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

1. A method of modulating angiogenesis in a mammal comprising administering to the mammal a therapeutically effective amount of a Nod factor.

2. The method of modulating angiogenesis in a mammal comprising administering to the mammal a therapeutically effective amount of an oligosaccharide of formula I or a pharmaceutically acceptable salt thereof: 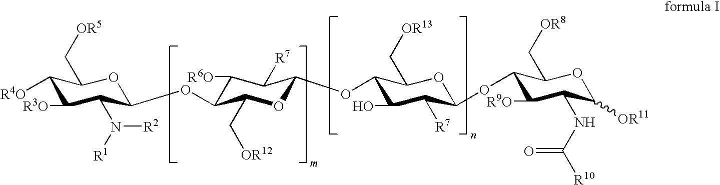
wherein:
R¹ is selected from hydrogen, —X-Alk or —X-Alk¹-Q-Y-Alk²;
wherein:
X is selected from —C(O)—, —C(NR^{N})—, —C(S)—, —SO₂—, —P(O)(OR^{N})— wherein R^{N} is hydrogen, hydroxy, amino, optionally substituted C₁₋₈alkyl, optionally substituted C₂₋₈alkenyl, optionally substituted C₂₋₈alkynyl, optionally substituted and optionally substituted aryl;
Alk is selected from an optionally substituted, straight chain or branched, alkyl, alkenyl or alkynyl group having from 2 to 30 carbon atoms;
Alk¹ is absent or present and is selected from an optionally substituted divalent C₁₋₁₀alkyl, optionally substituted divalent C₂₋₁₀alkenyl and optionally substituted divalent C₂₋₁₀alkynyl chain;
Q is absent or present and is selected from an optionally substituted divalent cycloalkyl, optionally substituted divalent cycloalkenyl, optionally substituted divalent heterocycle, optionally substituted divalent aryl or optionally substituted divalent heteroaryl ring system;
Y is absent or present and is selected from —NH—, —O—, —S—, —NHC(O)—, —C(O)NH—, NHSO₃—, —C(R^{G})═N—N—, —NHC(O)NH—, —NHC(S)NH—, —NHC(NH)NH—, —C(R^{G})═N, and —N═C(R)—, wherein R^{G} is hydrogen, optionally substituted C₁₋₆alkyl, optionally substituted arylC₁₋₄alkyl, optionally substituted aryl or optionally substituted heteroaryl, provided that both Q and Y are not simultaneously absent;
Alk² is absent or present and is selected from hydrogen, or an optionally substituted, straight chain or branched, alkyl, alkenyl or alkynyl group having from 1 to 30 carbon atoms;
R² is selected from hydrogen, C₁₋₄alkyl or R² can combine with R¹ and N to form an azide;
R³ and R⁴ are independently selected from hydrogen, carbamoyl and C₁₋₄acyl;
R⁵ is selected from hydrogen, fucopyranosyl, carbamoyl and C₁₋₄acyl;
R⁶ is selected from hydrogen, C₁₋₄acyl or a monosaccharide;
R⁷ is independently selected from an acetamide or a hydroxyl group;
R⁸ is selected from hydrogen, sulphonato, C₁₋₄acyl or a monosaccharide;
R⁹ is selected from hydrogen or a monosaccharide;
R¹⁰ is selected from hydrogen or optionally substituted C₁₋₄alkyl;
R¹¹ is selected from hydrogen, a monosaccharide, glycerol, C₁₋₄acyl or C₁₋₄alkyl;
R¹² is selected from hydrogen, fucopyranosyl or C₁₋₄acyl;
R¹³ is independently selected from hydrogen or fucopyranosyl;
m is an integer selected from 0 and 1;
n is an integer selected from 0 to 3; and
where the reducing end sugar ring is in open chain or ring closed form.

3. The method according to claim 2 wherein R¹ is hydrogen.

4. The method according to claim 2 wherein R¹ is —X-Alk and wherein Alk is selected from an optionally substituted, straight chain or branched, alkyl, alkenyl or alkynyl group having from 5 to 25 carbon atoms.

5. The method according to claim 4 wherein Alk is selected from an optionally substituted, straight chain or branched, alkyl, alkenyl or alkynyl group having from 10 to 25 carbon atoms.

6. The method according to claim 5 wherein Alk is selected from an optionally substituted, straight chain or branched, alkyl, alkenyl or alkynyl group having from 14 to 22 carbon atoms.

7. The method according to claim 4 wherein X is —C(O)—.

8. The method according to claim 2 wherein m is 1 and n is an integer selected from 1 to 2.

9. The method according to claim 2 wherein R¹ is —X-Alk¹-Q-Y-Alk² and wherein X is —C(O)—, Alk¹ is selected from divalent C₁₋₄alkyl or is absent, Q is selected from optionally substituted divalent aryl or optionally substituted divalent heteroaryl, Y is selected from —O—, —NH—, —S—, —NHC(O)—, or —C(O)NH—, and Alk² is an optionally substituted C₁₋₂₅alkyl or C₁₋₂₄alkenyl group.

10-11. (canceled)

12. The method according to claim 2 wherein R² is hydrogen.

13. The method according to claim 2 wherein R³, R⁴ and R⁵ are independently selected from hydrogen, carbamoyl or acetyl.

14-15. (canceled)

16. The method according to claim 2 wherein R⁶ is hydrogen.

17. The method according to claim 2 wherein R⁷ is an acetamide.

18. The method according to claim 2 wherein R⁸ is selected from hydrogen, sulphonato, C₁₋₄acyl, an unsubstituted monosaccharide, or a substituted monosaccharide of formula III: 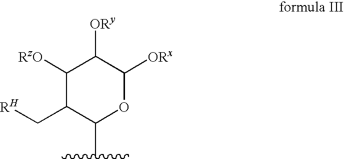
wherein:
R^{x} is selected from hydrogen, C₁₋₄alkyl or C₁₋₄acyl;
R^{y} is selected from hydrogen, sulphonato or C₁₋₄acyl;
R^{z} is selected from hydrogen, C₁₋₄alkyl or C₁₋₄acyl; and
R^{H} is selected from H or OR^{P}, wherein R^{P} is selected from hydrogen, C₁₋₄alkyl or C₁₋₄acyl.

19. The method according claim 2 wherein R⁸ is selected from hydrogen, arabinosyl, sulphonato, C₁₋₄acyl or a substituted monosaccharide of formula IV: 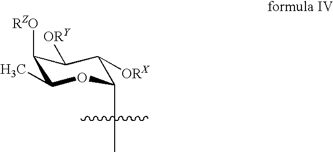
wherein:
R^{x} is selected from hydrogen, C₁₋₄alkyl or C₁₋₄acyl;
R^{y} is selected from hydrogen, sulphonato or C₁₋₄acyl; and
R^{z} is selected from hydrogen, C₁₋₄alkyl or C₁₋₄acyl.

20. The method according to claim 19 wherein R⁸ is selected from sulphonato or a substituted monosaccharide of formula IV wherein R^{z} is selected from acetyl or hydrogen, R is hydrogen, and R is selected from hydrogen or methyl.

21-29. (canceled)

30. The method according to claim 2 wherein R² is hydrogen or C₁₋₄alkyl; R³, R⁴ and R⁵ are independently selected from hydrogen, carbamoyl and C₁₋₄acyl; R⁶ is hydrogen, C₁₋₄acyl or α-L-fucopyranosyl; each R⁷ is independently selected from an acetamide or a hydroxyl group; R⁸ is hydrogen, arabinosyl, sulphonato, C₁₋₄acyl or a substituted monosaccharide of formula IV: 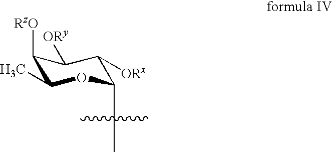
wherein:
R^{x} is hydrogen or C₁₋₄acyl, R^{y} is hydrogen, sulphonato or C₁₋₄acyl, and R^{z} is hydrogen, C₁₋₄alkyl or C₁₋₄acyl;
R⁹ is hydrogen, α-L-fucosyl or arabinosyl;
R¹⁰ is hydrogen, methyl or substituted methyl;
R¹¹ is hydrogen, mannosyl, glycerol or C₁₋₄alkyl;
R¹² is hydrogen or C₁₋₄acyl;
m is the integer 1; and
n is an integer selected from 1 or 2.

31. The method according to claim 2 wherein the oligosaccharide is of formula V 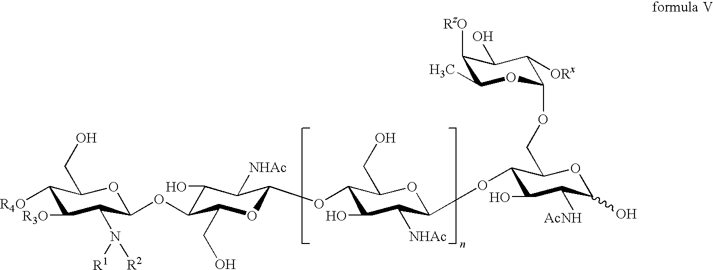
wherein:
R¹ is selected from hydrogen, —X-Alk or —X-Alk¹-Q-Y-Alk²;
R² is selected from hydrogen or methyl;
R³ and R⁴ are independently selected from hydrogen and carbamoyl;
R^{z} is selected from hydrogen or acetyl;
R^{x} is selected from hydrogen or methyl; and n is an integer selected from 1 or 2.

32-34. (canceled)

35. The method according to claim 2 wherein the oligosaccharide is of formula VI: 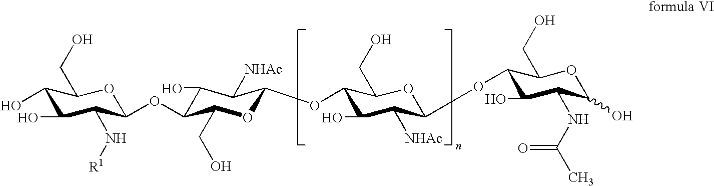
wherein:
R¹ is selected from hydrogen, —X-Alk or —X-Alk¹-Q-Y-Alk²; and n is an integer selected from 1 or 2.

36. The method according to claim 2 wherein the oligosaccharide is of formula VII: 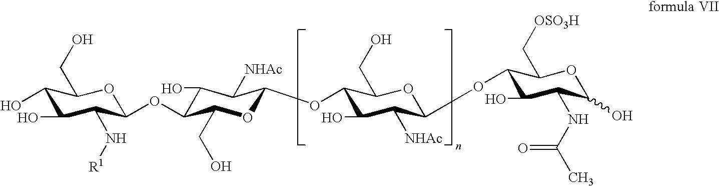
wherein:
R¹ is selected from hydrogen, —X-Alk or —X-Alk¹-Q-Y-Alk²; and
n is an integer selected from 1 or 2.

37. The method according to claim 2 wherein the oligosaccharide is of formula VIII: 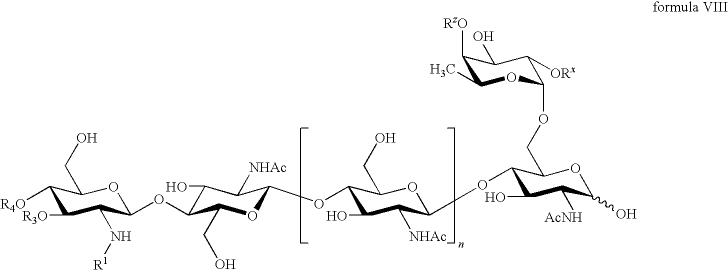
wherein:
R¹ is —X-Alk¹-Q-Y-Alk²;
X is selected from —C(O)—, —C(NR^{N})—, —C(S)—, —SO₂—, —P(O)(OR^{N})— wherein R^{N} is hydrogen, hydroxy, amino, optionally substituted C₁₋₈alkyl, optionally substituted C₂₋₈alkenyl, optionally substituted C₂₋₈alkynyl, optionally substituted C₁₋₄alkylaryl, and optionally substituted aryl;
Alk¹ is absent or present and is selected from an optionally substituted divalent C₁₋₁₀alkyl, optionally substituted divalent C₂₋₁₀alkenyl and optionally substituted divalent C₂₋₁₀alkynyl chain;
Q is absent or present and is selected from an optionally substituted divalent cycloalkyl, optionally substituted divalent cycloalkenyl, optionally substituted divalent heterocycle, optionally substituted divalent aryl or optionally substituted divalent heteroaryl ring system;
Y is absent or present and is selected from —NH—, —O—, —S—, —NHC(O)—, —C(O)NH—, NHSO₃—, —C(R^{G})═N—N—, —NHC(O)NH—, —NHC(S)NH—, —NHC(NH)NH—, —C(R^{G})═N—, and —N═C(R)—, wherein R^{G} is hydrogen, optionally substituted C₁₋₆alkyl, optionally substituted arylC₁₋₄alkyl, optionally substituted aryl or optionally substituted heteroaryl;
Alk² is absent or present and is selected from an optionally substituted, straight chain or branched, alkyl, alkenyl or alkynyl group having from 1 to 30 carbon atoms; and
n is an integer selected from 1 or 2.

38-39. (canceled)

40. The method according to claim 1 wherein the Nod factor is neutral, or does not have a charge, positive or negative, of greater magnitude than 1.

41. A method of preventing or treating an angiogenesis associated disorder comprising administering to a subject in a need of such treatment a therapeutically effective amount of a Nod-factor as defined in claim 1.

42. (canceled)

43. The method according to claim 41 wherein said therapeutically effective amount is an amount effective to inhibit primary tumor formation and metastasis in solid tumors, said tumors being associated with a cancer selected from rhabdomyosarcomas, retinoblastoma, Ewing sarcoma, neuroblastoma, osteosarcoma, colon, prostate, head and neck, breast, bladder, liver, pancreatic lung, CNS, Paget's disease and blood-born tumors such as leukemia and hemangiona.

44. The method according to claim 43 wherein the Nod factor is combined with at least one additional anti-cancer, anti-metastatic or antineoplastic agent.

45-64. (canceled)

65. An angiogenesis modulating composition comprising a Nod-factor as defined in claim 1.

66. An angiogenesis modulating composition comprising an oligosaccharide or salt of formula I as defined in claim 2.

67. The method of claim 2 wherein the oligosaccharide or salt is neutral, or does not have a charge, positive or negative, of greater magnitude than 1.

68. A method of preventing or treating an angiogenesis associated disorder comprising administering to a subject in a need of such treatment a therapeutically effective amount of an oligosaccharide or salt of formula I as defined in claim 2.

69. The method according to claim 68 wherein said therapeutically effective amount is an amount effective to inhibit primary tumor formation and metastasis in solid tumors, said tumors being associated with a cancer selected from rhabdomyosarcomas, retinoblastoma, Ewing sarcoma, neuroblastoma, osteosarcoma, colon, prostate, head and neck, breast, bladder, liver, pancreatic, lung, CNS, Paget's disease and blood-born tumors such as leukemia and hemangiona.

70. The method according to claim 69 wherein the oligosaccharide or salt is combined with at least one additional anti-cancer, anti-metastatic or antineoplastic agent.
